(19) United States
(12) Patent Application Publication
(10) Pub. No.: US 2022/0051276 A1
(43) Pub. Date: Feb. 17, 2022

(54) Data Analytics System, Method and Program Product for Processing Health Insurance Claims and Targeted Advertisement-Based Healthcare Management

(71) Applicant: Enzo Zelocchi, West Hollywood (IT)

(72) Inventor: Enzo Zelocchi, West Hollywood (IT)

(21) Appl. No.: 17/512,611

(22) Filed: Oct. 27, 2021

Related U.S. Application Data
(63) Continuation-in-part of application No. 16/509,477, filed on Jul. 11, 2019, now abandoned.

(51) Int. Cl. G06Q 30/02; G16H 10/60; G16H 80/00

(52) U.S. Cl. CPC G06Q 30/0208; G06Q 30/0251; G06Q 2220/00; G16H 10/60; G16H 80/00; G06Q 30/0203

(57) ABSTRACT

An all-inclusive blockchain-based program is provided to transform and streamline personal medical records, history and documentation of a participant in a singular portal to: seek appointments with medical practitioners; streamline flow of medical diagnostics; provide checks and balances to counteract improper medical exams, tests, over-prescription medication; and reward users with usable credits. Advertising content, paid for by a private-party advertiser, is provided to the participant to provide additional credit for consumption of the advertising content. Organ donation is coordinated immediately at least partially based on the transformed medical records of the blockchain-based program, as is facilitation of the participant to purchase medication. Organized and/or tracked shipment and a recycling program for the medication are also available. Ability to process and expedite with absolute transparency every single health insurance claim made by a citizen.

### CROSS-REFERENCE TO RELATED APPLICATIONS

The present continuation-in-part patent application claims priority benefit under 35 U.S.C. 120 of the U.S. nonprovisional patent application Ser. No. 16/509,477 entitled “DATA ANALYTICS SYSTEM, METHOD AND PROGRAM PRODUCT FOR PROCESSING HEALTH INSURANCE CLAIMS AND TARGETED ADVERTISEMENT-BASED HEALTHCARE MANAGEMENT”, filed on Jul. 11, 2019. The contents of this/these related patent application(s) is/are incorporated herein by reference for all purposes to the extent that such subject matter is not inconsistent herewith or limiting hereof.

### RELATED CO-PENDING U.S. PATENT APPLICATIONS

The following related U.S. patent application(s), submitted by at least one of the present Applicant(s)/Inventor(s) is/(are) recently co-pending:

U.S. utility patent application Ser. No. 16/509,477, entitled “DATA ANALYTICS SYSTEM, METHOD AND PROGRAM PRODUCT FOR PROCESSING HEALTH INSURANCE CLAIMS AND TARGETED ADVERTISEMENT-BASED HEALTHCARE MANAGEMENT”, submitted to the United States Patent and Trademark Office (USPTO) on Jul. 11, 2019.

### COPYRIGHT NOTICE

A portion of the disclosure of this patent document contains material that is subject to copyright protection by the author thereof. The copyright owner has no objection to the facsimile reproduction by anyone of the patent document or patent disclosure for the purposes of referencing as patent prior art, as it appears in the Patent and Trademark Office, patent file or records, but otherwise reserves all copyright rights whatsoever.

### BACKGROUND OF THE RELEVANT PRIOR ART

One or more embodiments of the invention generally relate to providing a universal system to optimize the offering of healthcare related services, including health insurance, identification of appropriate doctors, and the provision of related healthcare services. Utilization of advanced computer-science related technologies such as machine learning, artificial intelligence, and blockchain result in enhanced efficiency regarding patient-to-provider communication, where costs associated with implementation and ongoing upkeep of system operations are at least partially offset by private party advertising targeted to identified patients and/or consumers based on their respective needs.

More particularly, certain embodiments of the invention relate to providing affordable and accessible healthcare to low- and middle-income consumers and to actualize a system allowing participants to handle their respective healthcare-related bills promptly and conveniently. Some embodiments may provide means for processing and expediting health insurance claims. Participants may access different healthcare related sub-systems and/or platforms via a single centralized database that also allows for the confidential sharing of health-related information and records with doctors, other healthcare providers, and healthcare establishments, such as hospitals, worldwide. Disclosed systems and methods of use thereof further provide for addressing patient and/or consumer-related logistical needs, such as for the booking of doctors' appointments, switching between various healthcare related policies, the recycling of unused medication, and obtaining optimal pricing for doctors, medications, hospitals, etc.

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon. Systems and methods of use thereof may be known relating to implementing medical evaluation particular machine learning workflows and processes. Also, computer-implemented methods systems, and computer-readable storage mediums may have been provided for use with a clinical support system for identifying and providing information regarding causative relationship association between individual patient attributes and one or more adverse events. Systems, methods and devices for personal medical care, intelligent analysis and diagnosis may also be known, as are systems and methods for developing a comprehensive health profile.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 illustrates an exemplary schematic diagram of a system of a virtual intelligent healthcare-related services system;

FIG. 2 illustrates an exemplary schematic diagram of a system of a virtual intelligent healthcare-related services system;

FIG. 3 illustrates an exemplary flow diagram of a healthcare-related services system directed to integration of a virtual assistant and wearable technology;

FIG. 4 illustrates an exemplary schematic diagram of a system of a virtual intelligent healthcare-related services system;

FIG. 5 illustrates an example “cloud”-based system of a virtual intelligent healthcare-related services system;

FIG. 6 illustrates an example notification system of a virtual intelligent healthcare-related services system;

FIG. 7 illustrates an example organ tracking and notification system of a virtual intelligent healthcare-related services system;

FIG. 8 illustrates an example “cloud”-based system of a virtual intelligent healthcare-related services system;

FIG. 9 illustrates an example encryption system of a virtual intelligent healthcare-related services system;

FIG. 10 illustrates an exemplary schematic diagram of a “cloud”-based system of a virtual intelligent healthcare-related services system;

FIG. 11 illustrates an example “cloud”-based system of a virtual intelligent healthcare-related services system;

FIG. 12 illustrates an example system integrating wearable technology and robotics into the virtual intelligent healthcare-related services system;

FIG. 13 illustrates an example system integrating wearable technology and robotics into the virtual intelligent healthcare-related services system;

FIG. 14 illustrates an example “cloud”-based system implementing machine learning and microservices of a virtual intelligent healthcare-related services system;

FIG. 15 illustrates an example “cloud”-based system of a virtual intelligent healthcare-related services system;

FIG. 16 illustrates an example “cloud”-based system of a virtual intelligent healthcare-related services system;

FIG. 17 illustrates an example notification system for fraud prevention of a virtual intelligent healthcare-related services system;

FIG. 18 illustrates a block diagram depicting an exemplary client/server system which may be used by an exemplary web-enabled/networked embodiment of the present invention;

FIG. 19 illustrates a block diagram depicting a conventional client/server communication system, which may be used by an exemplary web-enabled/networked embodiment of the present invention;

FIG. 20 illustrates a flowchart of an exemplary method that uses blockchain technologies to track, analyze, store, and expedite personal medical records, history and documentation, in accordance with an embodiment of the present invention;

FIG. 21 illustrates a flowchart of an exemplary method of a subroutine of the embodiment that uses blockchain technologies to track, analyze, store, and expedite personal medical records, history and documentation, in accordance with an embodiment of the present invention;

FIG. 22 illustrates a flowchart of an exemplary method of a subroutine of the embodiment that uses blockchain technologies to track, analyze, store, and expedite personal medical records, history and documentation, in accordance with an embodiment of the present invention;

FIG. 23 illustrates an extended flowchart of additional embodiments of the exemplary method to make medical diagnostics more accurate and accessible by using machine learning to predict what will happen within based on symptoms and individual genetics, in accordance with an embodiment of the present invention; and

FIG. 24 illustrates an extended flowchart of additional embodiments of the exemplary method to make medical diagnostics more accurate and accessible by using machine learning to predict what will happen within based on symptoms and individual genetics, in accordance with an embodiment of the present invention.

FIG. 25 illustrates an exemplary embodiment of the integrated graphic user interface.

FIG. 26 illustrates an exemplary embodiment of the integrated graphic user interface.

FIG. 27 illustrates an exemplary embodiment of the integrated graphic user interface.

FIG. 28 illustrates an exemplary embodiment of the integrated graphic user interface.

Unless otherwise indicated illustrations in the figures are not necessarily drawn to scale.

### DETAILED DESCRIPTION OF SOME EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

All words of approximation as used in the present disclosure and claims should be construed to mean “approximate,” rather than “perfect,” and may accordingly be employed as a meaningful modifier to any other word, specified parameter, quantity, quality, or concept. Words of approximation, include, yet are not limited to terms such as “substantial”, “nearly”, “almost”, “about”, “generally”, “largely”, “essentially”, “closely approximate”, etc.

As will be established in some detail below, it is well settled law, as early as 1939, that words of approximation are not indefinite in the claims even when such limits are not defined or specified in the specification.

For example, see Ex parte Mallory, 52 USPQ 297, 297 (Pat. Off. Bd. App. 1941) where the court said “The examiner has held that most of the claims are inaccurate because apparently the laminar film will not be entirely eliminated. The claims specify that the film is “substantially” eliminated and for the intended purpose, it is believed that the slight portion of the film which may remain is negligible. We are of the view, therefore, that the claims may be regarded as sufficiently accurate.”

Note that claims need only “reasonably apprise those skilled in the art” as to their scope to satisfy the definiteness requirement. See Energy Absorption Sys., Inc. v. Roadway Safety Servs., Inc., Civ. App. 96-1264, slip op. at 10 (Fed. Cir. Jul. 3, 1997) (unpublished) Hybridtech v. Monoclonal Antibodies, Inc., 802 F.2d 1367, 1385, 231 USPQ 81, 94 (Fed. Cir. 1986), cert. denied, 480 U.S. 947 (1987). In addition, the use of modifiers in the claim, like “generally” and “substantial,” does not by itself render the claims indefinite. See Seattle Box Co. v. Industrial Crating & Packing, Inc., 731 F.2d 818, 828-29, 221 USPQ 568, 575-76 (Fed. Cir. 1984).

Moreover, the ordinary and customary meaning of terms like “substantially” includes “reasonably close to: nearly, almost, about”, connoting a term of approximation. See In re Frye, Appeal No. 2009-006013, 94 USPQ2d 1072, 1077, 2010 WL 889747 (B.P.A.I. 2010) Depending on its usage, the word “substantially” can denote either language of approximation or language of magnitude. Deering Precision Instruments, L.L.C. v. Vector Distribution Sys., Inc., 347 F.3d 1314, 1323 (Fed. Cir. 2003) (recognizing the “dual ordinary meaning of th[e] term [“substantially”] as connoting a term of approximation or a term of magnitude”). Here, when referring to the “substantially halfway” limitation, the Specification uses the word “approximately” as a substitute for the word “substantially” (Fact 4). (Fact 4). The ordinary meaning of “substantially halfway” is thus reasonably close to or nearly at the midpoint between the forwardmost point of the upper or outsole and the rearwardmost point of the upper or outsole.

Similarly, the term ‘substantially’ is well recognized in case law to have the dual ordinary meaning of connoting a term of approximation or a term of magnitude. See Dana Corp. v. American Axle & Manufacturing, Inc., Civ. App. 04-1116, 2004 U.S. App. LEXIS 18265, *13-14 (Fed. Cir. Aug. 27, 2004) (unpublished). The term “substantially” is commonly used by claim drafters to indicate approximation. See Cordis Corp. v. Medtronic AVE Inc., 339 F.3d 1352, 1360 (Fed. Cir. 2003) (“The patents do not set out any numerical standard by which to determine whether the thickness of the wall surface is ‘substantially uniform.’ The term ‘substantially,’ as used in this context, denotes approximation. Thus, the walls must be of largely or approximately uniform thickness.”); see also Deering Precision Instruments, LLC v. Vector Distribution Sys., Inc., 347 F.3d 1314, 1322 (Fed. Cir. 2003); Epcon Gas Sys., Inc. v. Bauer Compressors, Inc., 279 F.3d 1022, 1031 (Fed. Cir. 2002). We find that the term “substantially” was used in just such a manner in the claims of the patents-in-suit: “substantially uniform wall thickness” denotes a wall thickness with approximate uniformity.

It should also be noted that such words of approximation as contemplated in the foregoing clearly limits the scope of claims such as saying ‘generally parallel’ such that the adverb ‘generally’ does not broaden the meaning of parallel. Accordingly, it is well settled that such words of approximation as contemplated in the foregoing (e.g., like the phrase ‘generally parallel’) envisions some amount of deviation from perfection (e.g., not exactly parallel), and that such words of approximation as contemplated in the foregoing are descriptive terms commonly used in patent claims to avoid a strict numerical boundary to the specified parameter. To the extent that the plain language of the claims relying on such words of approximation as contemplated in the foregoing are clear and uncontradicted by anything in the written description herein or the figures thereof, it is improper to rely upon the present written description, the figures, or the prosecution history to add limitations to any of the claim of the present invention with respect to such words of approximation as contemplated in the foregoing. That is, under such circumstances, relying on the written description and prosecution history to reject the ordinary and customary meanings of the words themselves is impermissible. See, for example, Liquid Dynamics Corp. v. Vaughan Co., 355 F.3d 1361, 69 USPQ2d 1595, 1600-01 (Fed. Cir. 2004). The plain language of phrase 2 requires a “substantial helical flow.” The term “substantial” is a meaningful modifier implying “approximate,” rather than “perfect.” In Cordis Corp. v. Medtronic AVE, Inc., 339 F.3d 1352, 1361 (Fed. Cir. 2003), the district court imposed a precise numeric constraint on the term “substantially uniform thickness.” We noted that the proper interpretation of this term was “of largely or approximately uniform thickness” unless something in the prosecution history imposed the “clear and unmistakable disclaimer” needed for narrowing beyond this simple-language interpretation. Id. In Anchor Wall Systems v. Rockwood Retaining Walls, Inc., 340 F.3d 1298, 1311 (Fed. Cir. 2003)” Id. at 1311. Similarly, the plain language of claim 1 requires neither a perfectly helical flow nor a flow that returns precisely to the center after one rotation (a limitation that arises only as a logical consequence of requiring a perfectly helical flow).

The reader should appreciate that case law generally recognizes a dual ordinary meaning of such words of approximation, as contemplated in the foregoing, as connoting a term of approximation or a term of magnitude; e.g., see Deering Precision Instruments, L.L.C. v. Vector Distrib. Sys., Inc., 347 F.3d 1314, 68 USPQ2d 1716, 1721 (Fed. Cir. 2003), cert. denied, 124 S. Ct. 1426 (2004) where the court was asked to construe the meaning of the term “substantially” in a patent claim. Also see Epcon, 279 F.3d at 1031 (“The phrase ‘substantially constant’ denotes language of approximation, while the phrase ‘substantially below’ signifies language of magnitude, i.e., not insubstantial.”). Also, see, e.g., Epcon Gas Sys., Inc. v. Bauer Compressors, Inc., 279 F.3d 1022 (Fed. Cir. 2002) (construing the terms “substantially constant” and “substantially below”); Zodiac Pool Care, Inc. v. Hoffinger Indus., Inc., 206 F.3d 1408 (Fed. Cir. 2000) (construing the term “substantially inward”); York Prods., Inc. v. Cent. Tractor Farm & Family Ctr., 99 F.3d 1568 (Fed. Cir. 1996) (construing the term “substantially the entire height thereof”); Tex. Instruments Inc. v. Cypress Semiconductor Corp., 90 F.3d 1558 (Fed. Cir. 1996) (construing the term “substantially in the common plane”). In conducting their analysis, the court instructed to begin with the ordinary meaning of the claim terms to one of ordinary skill in the art. Prima Tek, 318 F.3d at 1148. Reference to dictionaries and our cases indicates that the term “substantially” has numerous ordinary meanings. As the district court stated, “substantially” can mean “significantly” or “considerably.” The term “substantially” can also mean “largely” or “essentially.” Webster's New 20th Century Dictionary 1817 (1983).

Words of approximation, as contemplated in the foregoing, may also be used in phrases establishing approximate ranges or limits, where the end points are inclusive and approximate, not perfect; e.g., see AK Steel Corp. v. Sollac, 344 F.3d 1234, 68 USPQ2d 1280, 1285 (Fed. Cir. 2003) where it where the court said [W]e conclude that the ordinary meaning of the phrase “up to about 10%” includes the “about 10%” endpoint. As pointed out by AK Steel, when an object of the preposition “up to” is nonnumeric, the most natural meaning is to exclude the object (e.g., painting the wall up to the door). On the other hand, as pointed out by Sollac, when the object is a numerical limit, the normal meaning is to include that upper numerical limit (e.g., counting up to ten, seating capacity for up to seven passengers). Because we have here a numerical limit—“about 10%”—the ordinary meaning is that that endpoint is included.

In the present specification and claims, a goal of employment of such words of approximation, as contemplated in the foregoing, is to avoid a strict numerical boundary to the modified specified parameter, as sanctioned by Pall Corp. v. Micron Separations, Inc., 66 F.3d 1211, 1217, 36 USPQ2d 1225, 1229 (Fed. Cir. 1995) where it states “It is well established that when the term “substantially” serves reasonably to describe the subject matter so that its scope would be understood by persons in the field of the invention, and to distinguish the claimed subject matter from the prior art, it is not indefinite.” Likewise see Verve LLC v. Crane Cams Inc., 311 F.3d 1116, 65 USPQ2d 1051, 1054 (Fed. Cir. 2002). Expressions such as “substantially” are used in patent documents when warranted by the nature of the invention, in order to accommodate the minor variations that may be appropriate to secure the invention. Such usage may well satisfy the charge to “particularly point out and distinctly claim” the invention, 35 U.S.C. § 112, and indeed may be necessary in order to provide the inventor with the benefit of his invention. In Andrew Corp. v. Gabriel Elecs. Inc., 847 F.2d 819, 821-22, 6 USPQ2d 2010, 2013 (Fed. Cir. 1988) the court explained that usages such as “substantially equal” and “closely approximate” may serve to describe the invention with precision appropriate to the technology and without intruding on the prior art. The court again explained in Ecolab Inc. v. Envirochem, Inc., 264 F.3d 1358, 1367, 60 USPQ2d 1173, 1179 (Fed. Cir. 2001) that “like the term ‘about,’ the term ‘substantially’ is a descriptive term commonly used in patent claims to ‘avoid a strict numerical boundary to the specified parameter, see Ecolab Inc. v. Envirochem Inc., 264 F.3d 1358, 60 USPQ2d 1173, 1179 (Fed. Cir. 2001) where the court found that the use of the term “substantially” to modify the term “uniform” does not render this phrase so unclear such that there is no means by which to ascertain the claim scope.

Similarly, other courts have noted that like the term “about,” the term “substantially” is a descriptive term commonly used in patent claims to “avoid a strict numerical boundary to the specified parameter.”; e.g., see Pall Corp. v. Micron Seps., 66 F.3d 1211, 1217, 36 USPQ2d 1225, 1229 (Fed. Cir. 1995); see, e.g., Andrew Corp. v. Gabriel Elecs. Inc., 847 F.2d 819, 821-22, 6 USPQ2d 2010, 2013 (Fed. Cir. 1988) (noting that terms such as “approach each other,” “close to,” “substantially equal,” and “closely approximate” are ubiquitously used in patent claims and that such usages, when serving reasonably to describe the claimed subject matter to those of skill in the field of the invention, and to distinguish the claimed subject matter from the prior art, have been accepted in patent examination and upheld by the courts). In this case, “substantially” avoids the strict 100% nonuniformity boundary.

Indeed, the foregoing sanctioning of such words of approximation, as contemplated in the foregoing, has been established as early as 1939, see Ex parte Mallory, 52 USPQ 297, 297 (Pat. Off. Bd. App. 1941) where, for example, the court said “the claims specify that the film is “substantially” eliminated and for the intended purpose, it is believed that the slight portion of the film which may remain is negligible. We are of the view, therefore, that the claims may be regarded as sufficiently accurate.” Similarly, In re Hutchison, 104 F.2d 829, 42 USPQ 90, 93 (C.C.P.A. 1939) the court said “It is realized that “substantial distance” is a relative and somewhat indefinite term, or phrase, but terms and phrases of this character are not uncommon in patents in cases where, according to the art involved, the meaning can be determined with reasonable clearness.”

Hence, for at least the forgoing reason, Applicant submits that it is improper for any examiner to hold as indefinite any claims of the present patent that employ any words of approximation.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will be described in detail below with reference to embodiments thereof as illustrated in the accompanying drawings.

References to a “device,” an “apparatus,” a “system,” etc., in the preamble of a claim should be construed broadly to mean “any structure meeting the claim terms” exempt for any specific structure(s)/type(s) that has/(have) been explicitly disavowed or excluded or admitted/implied as prior art in the present specification or incapable of enabling an object/aspect/goal of the invention. Furthermore, where the present specification discloses an object, aspect, function, goal, result, or advantage of the invention that a specific prior art structure and/or method step is similarly capable of performing yet in a very different way, the present invention disclosure is intended to and shall also implicitly include and cover additional corresponding alternative embodiments that are otherwise identical to that explicitly disclosed except that they exclude such prior art structure(s)/step(s), and shall accordingly be deemed as providing sufficient disclosure to support a corresponding negative limitation in a claim claiming such alternative embodiment(s), which exclude such very different prior art structure(s)/step(s) way(s).

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicant hereby gives notice that new Claims may be formulated to such features and/or combinations of such features during the prosecution of the present Application or of any further Application derived therefrom.

### Definitions

References to “one embodiment,” “an embodiment,” “example embodiment,” “various embodiments,” “some embodiments,” “embodiments of the invention,” etc., may indicate that the embodiment(s) of the invention so described may include a particular feature, structure, or characteristic, but not every possible embodiment of the invention necessarily includes the particular feature, structure, or characteristic. Further, repeated use of the phrase “in one embodiment,” or “in an exemplary embodiment,” “an embodiment,” do not necessarily refer to the same embodiment, although they may. Moreover, any use of phrases like “embodiments” in connection with “the invention” are never meant to characterize that all embodiments of the invention must include the particular feature, structure, or characteristic, and should instead be understood to mean “at least some embodiments of the invention” include the stated particular feature, structure, or characteristic.

References to “user”, or any similar term, as used herein, may mean a human or non-human user thereof. Moreover, “user”, or any similar term, as used herein, unless expressly stipulated otherwise, is contemplated to mean users at any stage of the usage process, to include, without limitation, direct user(s), intermediate user(s), indirect user(s), and end user(s). The meaning of “user”, or any similar term, as used herein, should not be otherwise inferred or induced by any pattern(s) of description, embodiments, examples, or referenced prior-art that may (or may not) be provided in the present patent.

References to “end user”, or any similar term, as used herein, is generally intended to mean late stage user(s) as opposed to early stage user(s). Hence, it is contemplated that there may be a multiplicity of different types of “end user” near the end stage of the usage process. Where applicable, especially with respect to distribution channels of embodiments of the invention comprising consumed retail products/services thereof (as opposed to sellers/vendors or Original Equipment Manufacturers), examples of an “end user” may include, without limitation, a “consumer”, “buyer”, “customer”, “purchaser”, “shopper”, “enjoyer”, “viewer”, or individual person or non-human thing benefiting in any way, directly or indirectly, from use of or interaction, with some aspect of the present invention.

In some situations, some embodiments of the present invention may provide beneficial usage to more than one stage or type of usage in the foregoing usage process. In such cases where multiple embodiments targeting various stages of the usage process are described, references to “end user”, or any similar term, as used therein, are generally intended to not include the user that is the furthest removed, in the foregoing usage process, from the final user therein of an embodiment of the present invention.

Where applicable, especially with respect to retail distribution channels of embodiments of the invention, intermediate user(s) may include, without limitation, any individual person or non-human thing benefiting in any way, directly or indirectly, from use of, or interaction with, some aspect of the present invention with respect to selling, vending, Original Equipment Manufacturing, marketing, merchandising, distributing, service providing, and the like thereof.

References to “person”, “individual”, “human”, “a party”, “animal”, “creature”, or any similar term, as used herein, even if the context or particular embodiment implies living user, maker, or participant, it should be understood that such characterizations are sole by way of example, and not limitation, in that it is contemplated that any such usage, making, or participation by a living entity in connection with making, using, and/or participating, in any way, with embodiments of the present invention may be substituted by such similar performed by a suitably configured non-living entity, to include, without limitation, automated machines, robots, humanoids, computational systems, information processing systems, artificially intelligent systems, and the like. It is further contemplated that those skilled in the art will readily recognize the practical situations where such living makers, users, and/or participants with embodiments of the present invention may be in whole, or in part, replaced with such non-living makers, users, and/or participants with embodiments of the present invention.

Likewise, when those skilled in the art identify such practical situations where such living makers, users, and/or participants with embodiments of the present invention may be in whole, or in part, replaced with such non-living makers, it will be readily apparent in light of the teachings of the present invention how to adapt the described embodiments to be suitable for such non-living makers, users, and/or participants with embodiments of the present invention. Thus, the invention is thus to also cover all such modifications, equivalents, and alternatives falling within the spirit and scope of such adaptations and modifications, at least in part, for such non-living entities.

References to “healthcare” or “health-care” imply the maintenance or improvement of health via the prevention, diagnosis, and treatment of disease, illness, injury, and other physical and mental impairments in people. Health care is delivered by health professionals in allied health fields. Physicians and physician associates are a part of these health professionals. Dentistry, midwifery, nursing, medicine, optometry, audiology, pharmacy, psychology, occupational therapy, physical therapy and other health professions are all part of health care. It includes work done in providing primary care, secondary care, and tertiary care, as well as in public health.

Access to health care may vary across countries, communities, and individuals, largely influenced by social and economic conditions as well as health policies. Providing health care services means “the timely use of personal health services to achieve the best possible health outcomes”.[ Source: Access to Health Care in America. The National Academies Press, US National Academies of Science, Engineering and Medicine. 1993.] Factors to consider in terms of healthcare access include financial limitations (such as insurance coverage), geographic barriers (such as additional transportation costs, possibility to take paid time off of work to use such services), and personal limitations (lack of ability to communicate with healthcare providers, poor health literacy, low income). [Source: “Healthcare Access in Rural Communities Introduction”. Rural Health Information Hub. 2019. Retrieved 2019 Jun. 14.] Limitations to health care services affects negatively the use of medical services, efficacy of treatments, and overall outcome (well-being, mortality rates).

Health care systems are organizations established to meet the health needs of targeted populations. According to the World Health Organization (WHO), a well-functioning health care system requires a financing mechanism, a well-trained and adequately paid workforce, reliable information on which to base decisions and policies, and well-maintained health facilities to deliver quality medicines and technologies. [Source: “Health Topics: Health Systems”. www.who.int. World Health Organization. Retrieved 2013 Nov. 24.]

An efficient health care system can contribute to a significant part of a country's economy, development and industrialization. Health care is conventionally regarded as an important determinant in promoting the general physical and mental health and well-being of people around the world. An example of this was the worldwide eradication of smallpox in 1980, declared by the WHO as the first disease in human history to be completely eliminated by deliberate health care interventions. [Source: World Health Organization. Anniversary of smallpox eradication. Geneva, 18 Jun. 2010.]

References to “healthcare industry” imply an aggregation and integration of sectors within the economic system that provides goods and services to treat patients with curative, preventive, rehabilitative, and palliative care. It includes the generation and commercialization of goods and services lending themselves to maintaining and re-establishing health. [Source: “10 Jahre Nationale branchenkonferenz Gesundheitswirtschaft—Ausgewählte Ergebnisse p. 4” (PDF). BioCon Valley GmbH. Retrieved 21 Aug. 2015.] The modern healthcare industry is divided into many sectors and depends on the interdisciplinary teams of trained professionals and paraprofessionals to meet health needs of individuals and populations. [Source: https://cns.utexas.edu/health-professions; retrieved on: Jul. 7, 2019; “Health Care Initiatives, Employment & Training Administration (ETA)—U.S. Department of Labor”; Doleta.gov. Retrieved Feb. 17, 2015.]

References to “health economics” imply a branch of economics concerned with issues related to efficiency, effectiveness, value and behavior in the production and consumption of health and healthcare. In broad terms, health economists study the functioning of healthcare systems and health-affecting behaviors such as smoking.

References to “health maintenance organization (“HMO”)” imply a medical insurance group that provides health services for a fixed annual fee.[ Source: “BBC News—G-I—Health Maintenance Organization/HMO”. news.bbc.co.uk. Retrieved 22 Mar. 2018.] It is an organization that provides or arranges managed care for health insurance, self-funded health care benefit plans, individuals, and other entities, acting as a liaison with health care providers (hospitals, doctors, etc.) on a prepaid basis. The Health Maintenance Organization Act of 1973 required employers with 25 or more employees to offer federally certified HMO options if the employer offers traditional healthcare options. [Source: Joseph L. Dorsey, “The Health Maintenance Organization Act of 1973 (P.L. 93-222) and Prepaid Group Practice Plan,” Medical Care, Vol. 13, No. 1, (January, 1975), pp. 1-9] Unlike traditional indemnity insurance, an HMO covers care rendered by those doctors and other professionals who have agreed by contract to treat patients in accordance with the HMO's guidelines and restrictions in exchange for a steady stream of customers. HMOs cover emergency care regardless of the health care provider's contracted status.

References to “medical imaging” imply a technique and process of creating visual representations of the interior of a body for clinical analysis and medical intervention, as well as visual representation of the function of some organs or tissues (physiology). Medical imaging seeks to reveal internal structures hidden by the skin and bones, as well as to diagnose and treat disease. Medical imaging also establishes a database of normal anatomy and physiology to make it possible to identify abnormalities. Although imaging of removed organs and tissues can be performed for medical reasons, such procedures are usually considered part of pathology instead of medical imaging. As a discipline and in its widest sense, it is part of biological imaging and incorporates radiology which uses the imaging technologies of X-ray radiography, magnetic resonance imaging, medical ultrasonography or ultrasound, endoscopy, elastography, tactile imaging, thermography, medical photography and nuclear medicine functional imaging techniques as positron emission tomography (PET) and Single-photon emission computed tomography (SPECT). Measurement and recording techniques which are not primarily designed to produce images, such as electroencephalography (EEG), magnetoencephalography (MEG), electrocardiography (ECG), and others represent other technologies which produce data susceptible to representation as a parameter graph vs. time or maps which contain data about the measurement locations. In a limited comparison, these technologies can be considered as forms of medical imaging in another discipline.

References to “disease” imply particular abnormal condition that negatively affects the structure or function of part or all of an organism, and that is not due to any external injury.[Source: “Disease” at Dorland's Medical Dictionary; White, Tim (19 Dec. 2014), “What is the Difference Between an ‘Injury’ and ‘Disease’ for Commonwealth Injury Claims?”, Tindall Gask Bentley. Archived from the original on 27 Oct. 2017. Retrieved on Nov. 6, 2017.] Diseases are often construed as medical conditions that are associated with specific symptoms and signs.[Source: “Disease” at Dorland's Medical Dictionary] A disease may be caused by external factors such as pathogens or by internal dysfunctions. For example, internal dysfunctions of the immune system can produce a variety of different diseases, including various forms of immunodeficiency, hypersensitivity, allergies and autoimmune disorders. In humans, disease is often used more broadly to refer to any condition that causes pain, dysfunction, distress, social problems, or death to the person afflicted, or similar problems for those in contact with the person. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases can affect people not only physically, but also mentally, as contracting and living with a disease can alter the affected person's perspective on life. Death due to disease is called death by natural causes. There are four main types of disease: infectious diseases, deficiency diseases, hereditary diseases (including both genetic diseases and non-genetic hereditary diseases), and physiological diseases. Diseases can also be classified in other ways, such as communicable versus non-communicable diseases. The deadliest diseases in humans are coronary artery disease (blood flow obstruction), followed by cerebrovascular disease and lower respiratory infections.[“What is the deadliest disease in the world?”. WHO; 16 May 2012; Archived from the original on 17 Dec. 2014; Retrieved on: Dec. 7, 2014.] In developed countries, the diseases that cause the most sickness overall are neuropsychiatric conditions, such as depression and anxiety.

References to “preventative healthcare” imply measures taken for disease prevention.[Source: Hugh R. Leavell and E. Gurney Clark as “the science and art of preventing disease, prolonging life, and promoting physical and mental health and efficiency. Leavell, H. R., & Clark, E. G. (1979). Preventive Medicine for the Doctor in his Community (3rd ed.). Huntington, N.Y.: Robert E. Krieger Publishing Company.] Just as health comprises a variety of physical and mental states, so do disease and disability, which are affected by environmental factors, genetic predisposition, disease agents, and lifestyle choices. Health, disease, and disability are dynamic processes which begin before individuals realize they are affected. Disease prevention relies on anticipatory actions that can be categorized as primal, [source: “New parents” secure a lifelong well-being for their offspring by refusing to be victims of societal stress during its primal period”. Primal Prevention][source: Primal Health Research Database, on {{cite web |url=https://web. archive. org/web/20180815043657/http://primalhealthresearch.com/glossary.php] primary, secondary, and tertiary prevention. [Source: Hugh R. Leavell and E. Gurney Clark as “the science and art of preventing disease, prolonging life, and promoting physical and mental health and efficiency. Leavell, H. R., & Clark, E. G. (1979). Preventive Medicine for the Doctor in his Community (3rd ed.). Huntington, N.Y.: Robert E. Krieger Publishing Company.]

References to “artificial intelligence” imply a intelligence demonstrated by machines, in contrast to the natural intelligence displayed by humans. Colloquially, the term “artificial intelligence” is often used to describe machines (or computers) that mimic “cognitive” functions that humans associate with the human mind, such as “learning” and “problem solving”. [Source: Russell, Stuart J.; Norvig, Peter (2009). Artificial Intelligence: A Modern Approach (3rd ed.). Upper Saddle River, N.J.: Prentice Hall.] As machines become increasingly capable, tasks considered to require “intelligence” are often removed from the definition of AI, a phenomenon known as the AI effect. [Source: McCorduck, Pamela (2004), Machines Who Think (2nd ed.), Natick, Mass.: A. K. Peters, Ltd.] A quip in Tesler's Theorem says “AI is whatever hasn't been done yet.” [Source: Maloof, Mark. “Artificial Intelligence: An Introduction, p. 37” (PDF)]. For instance, optical character recognition is frequently excluded from things considered to be AI, having become a routine technology. [Source: Schank, Roger C. (1991). “Where's the AI”. AI magazine. Vol. 12 no. 4. p. 38.] Modern machine capabilities generally classified as AI include successfully understanding human speech, [source: Russell, Stuart J.; Norvig, Peter (2009). Artificial Intelligence: A Modern Approach (3rd ed.). Upper Saddle River, N.J.: Prentice Hall.] competing at the highest level in strategic game systems (such as chess and Go),[source: https://deepmind.com/research/alphago/; retrieved on: Jul. 7, 2019] autonomously operating cars, intelligent routing in content delivery networks, and military simulations. Artificial intelligence can be classified into three different types of systems: analytical, human-inspired, and humanized artificial intelligence. [Source: Kaplan Andreas; Michael Haenlein (2018) Siri, Siri in my Hand, who's the Fairest in the Land? On the Interpretations, Illustrations and Implications of Artificial Intelligence, Business Horizons, 62(1)] Analytical AI has only characteristics consistent with cognitive intelligence; generating cognitive representation of the world and using learning based on past experience to inform future decisions. Human-inspired AI has elements from cognitive and emotional intelligence; understanding human emotions, in addition to cognitive elements, and considering them in their decision making. Humanized AI shows characteristics of all types of competencies (i.e., cognitive, emotional, and social intelligence), is able to be self-conscious and is self-aware in interactions with others.

References to “machine learning” imply the scientific study of algorithms and statistical models that computer systems use in order to perform a specific task effectively without using explicit instructions, relying on patterns and inference instead. It is seen as a subset of artificial intelligence. Machine learning algorithms build a mathematical model based on sample data, known as “training data”, in order to make predictions or decisions without being explicitly programmed to perform the task. [Source: The definition “without being explicitly programmed” is often attributed to Arthur Samuel, who coined the term “machine learning” in 1959, but the phrase is not found verbatim in this publication, and may be a paraphrase that appeared later. Confer “Paraphrasing Arthur Samuel (1959), the question is: How can computers learn to solve problems without being explicitly programmed?” in Koza, John R.; Bennett, Forrest H.; Andre, David; Keane, Martin A. (1996). Automated Design of Both the Topology and Sizing of Analog Electrical Circuits Using Genetic Programming. Artificial Intelligence in Design '96. Springer, Dordrecht. pp. 151-170; Bishop, C. M. (2006), Pattern Recognition and Machine Learning, Springer]. Machine learning algorithms are used in a wide variety of applications, such as email filtering, and computer vision, where it is infeasible to develop an algorithm of specific instructions for performing the task. Machine learning is closely related to computational statistics, which focuses on making predictions using computers. The study of mathematical optimization delivers methods, theory and application domains to the field of machine learning. Data mining is a field of study within machine learning, and focuses on exploratory data analysis through unsupervised learning.[Source: Bishop, C. M. (2006), Pattern Recognition and Machine Learning, Springer (stating that machine learning and pattern recognition “can be viewed as two facets of the same field.)][Source: Friedman, Jerome H. (1998). “Data Mining and Statistics: What's the connection?”; Computing Science and Statistics. 29 (1): 3-9] In its application across business problems, machine learning is also referred to as predictive analytics.

References to “blockchain” imply a growing list of records, called blocks, that are linked using cryptography. [Source: “Blockchains: The great chain of being sure about things”. The Economist. 31 Oct. 2015. Archived from the original on 3 Jul. 2016. Retrieved 18 Jun. 2016. “The technology behind bitcoin lets people who do not know or trust each other build a dependable ledger. This has implications far beyond the crypto currency”; Narayanan, Arvind; Bonneau, Joseph; Felten, Edward; Miller, Andrew; Goldfeder, Steven (2016). Bitcoin and cryptocurrency technologies: a comprehensive introduction. Princeton: Princeton University Press.] Each block contains a cryptographic hash of the previous block,[source: Narayanan, Arvind; Bonneau, Joseph; Felten, Edward; Miller, Andrew; Goldfeder, Steven (2016). Bitcoin and cryptocurrency technologies: a comprehensive introduction. Princeton: Princeton University Press.] a timestamp, and transaction data (generally represented as a Merkle tree). By design, a blockchain is resistant to modification of the data. It is “an open, distributed ledger that can record transactions between two parties efficiently and in a verifiable and permanent way”. [Source: Iansiti, Marco; Lakhani, Karim R. (January 2017). “The Truth About Blockchain”. Harvard Business Review. Harvard University. Archived from the original on 18 Jan. 2017. Retrieved 17 Jan. 2017. “The technology at the heart of bitcoin and other virtual currencies, blockchain is an open, distributed ledger that can record transactions between two parties efficiently and in a verifiable and permanent way.”] For use as a distributed ledger, a blockchain is typically managed by a peer-to-peer network collectively adhering to a protocol for inter-node communication and validating new blocks. Once recorded, the data in any given block cannot be altered retroactively without alteration of all subsequent blocks, which requires consensus of the network majority. Although blockchain records are not unalterable, blockchains may be considered secure by design and exemplify a distributed computing system with high Byzantine fault tolerance. Decentralized consensus has therefore been claimed with a blockchain.[ Source: Raval, Siraj (2016). “What Is a Decentralized Application?”. Decentralized Applications: Harnessing Bitcoin's Blockchain Technology. O'Reilly Media, Inc. pp. 1-2. ISBN 978-1-4919-2452-5. OCLC 968277125. Retrieved 6 Nov. 2016—via Google Books.]

References to “Medicare” imply a national health insurance program in the United States, begun in 1966 under the Social Security Administration (SSA) and now administered by the Centers for Medicare and Medicaid Services (CMS). It provides health insurance for Americans aged 65 and older, younger people with some disability status as determined by the Social Security Administration, as well as people with end stage renal disease and amyotrophic lateral sclerosis (ALS or Lou Gehrig's disease). In 2018, Medicare provided health insurance for over 59.9 million individuals—more than 52 million people aged 65 and older and about 8 million younger people. [Source: 2019 Annual Report of the Medicare Trustees (for the year 2018), Apr. 22, 2019]. On average, Medicare covers about half of healthcare expenses of those enrolled. Medicare is funded by a combination of a payroll tax, beneficiary premiums and surtaxes from beneficiaries, co-pays and deductibles, and general U.S. Treasury revenue.

References to “data management platform (“DMP”)” imply a technology platform used for collecting and managing data, mainly for digital marketing purposes. [Source: “What is a Data Management Platform?—What is a DMP?”. lotame.com. 22 May 2018. Retrieved on: Jul. 5, 2018]. It allows to generate audience segments, which are used to target specific users in online advertising campaigns. The DMP may use Big Data and Artificial Intelligence algorithms to process big data sets about users from various sources. DMP is used for organizing and monetizing data in Real-Time Bidding system by licensing it to global selling platforms (DSPs). This technology is constantly being developed by entities such as Nielsen and Oracle.

References to “genomics” imply an interdisciplinary field of biology focusing on the structure, function, evolution, mapping, and editing of genomes. A genome is an organism's complete set of DNA, including all of its genes. In contrast to genetics, which refers to the study of individual genes and their roles in inheritance, genomics aims at the collective characterization and quantification of all of an organism's genes, their interrelations and influence on the organism. [Source: https://www.who.int/genomics/geneticsVSgenomics/en/; retrieved on Jul. 7, 2019]. Genes may direct the production of proteins with the assistance of enzymes and messenger molecules. In turn, proteins make up body structures such as organs and tissues as well as control chemical reactions and carry signals between cells. Genomics also involves the sequencing and analysis of genomes through uses of high throughput DNA sequencing and bioinformatics to assemble and analyze the function and structure of entire genomes. [Source: National Human Genome Research Institute (8 Nov. 2010). “A Brief Guide to Genomics”. Genome.gov. Retrieved 2011 Dec. 3; Concepts of genetics (10th ed.). San Francisco: Pearson Education. 2012; Culver K W, Labow M A (8 Nov. 2002). “Genomics”. In Robinson R (ed.). Genetics. Macmillan Science Library. Macmillan Reference USA.] Advances in genomics have triggered a revolution in discovery-based research and systems biology to facilitate understanding of even the most complex biological systems such as the brain. [Source: Kadakkuzha B M, Puthanveettil S V (July 2013). “Genomics and proteomics in solving brain complexity”. Molecular BioSystems. 9 (7): 1807-21] The field also includes studies of intragenomic (within the genome) phenomena such as epistasis (effect of one gene on another), pleiotropy (one gene affecting more than one trait), heterosis (hybrid vigor), and other interactions between loci and alleles within the genome.

References to “medical diagnosis (“Dx” or “Dₛ”)” imply the process of determining which disease or condition explains a person's symptoms and signs. It is most often referred to as diagnosis with the medical context being implicit. The information required for diagnosis is typically collected from a history and physical examination of the person seeking medical care. Often, one or more diagnostic procedures, such as medical tests, are also done during the process. Sometimes posthumous diagnosis is considered a kind of medical diagnosis.

References to “virtual health assistants” imply a virtual and/or online-based messaging service or system directed to provide answers responsive to specific patient and/or customer inquiries. For instance, “disease-specific bots can answer queries regarding disease—for both patients and doctors, as well as other health professionals and patient relatives. A child health bot can answer questions about children's health for their parents, with information about a wide range of symptoms and illnesses fed into it.” [Source: https://chatbotslife.com/artificial-intelligence-based-virtual-health-assistants-the-new-disruptors-42e9f2b44d40; Retrieved on: Jul. 7, 2019].

Headings provided herein are for convenience and are not to be taken as limiting the disclosure in any way.

The enumerated listing of items does not imply that any or all of the items are mutually exclusive, unless expressly specified otherwise.

It is understood that the use of specific component, device and/or parameter names are for example only and not meant to imply any limitations on the invention. The invention may thus be implemented with different nomenclature/terminology utilized to describe the mechanisms/units/structures/components/devices/parameters herein, without limitation. Each term utilized herein is to be given its broadest interpretation given the context in which that term is utilized.

### Terminology

The following paragraphs provide definitions and/or context for terms found in this disclosure (including the appended claims):

“Comprising” And “contain” and variations of them—Such terms are open-ended and mean “including but not limited to”. When employed in the appended claims, this term does not foreclose additional structure or steps. Consider a claim that recites: “A memory controller comprising a system cache . . . .” Such a claim does not foreclose the memory controller from including additional components (e.g., a memory channel unit, a switch).

“Configured To.” Various units, circuits, or other components may be described or claimed as “configured to” perform a task or tasks. In such contexts, “configured to” or “operable for” is used to connote structure by indicating that the mechanisms/units/circuits/components include structure (e.g., circuitry and/or mechanisms) that performs the task or tasks during operation. As such, the mechanisms/unit/circuit/component can be said to be configured to (or be operable) for perform(ing) the task even when the specified mechanisms/unit/circuit/component is not currently operational (e.g., is not on). The mechanisms/units/circuits/components used with the “configured to” or “operable for” language include hardware—for example, mechanisms, structures, electronics, circuits, memory storing program instructions executable to implement the operation, etc. Reciting that a mechanism/unit/circuit/component is “configured to” or “operable for” perform(ing) one or more tasks is expressly intended not to invoke 35 U.S.C. .sctn.112, sixth paragraph, for that mechanism/unit/circuit/component. “Configured to” may also include adapting a manufacturing process to fabricate devices or components that are adapted to implement or perform one or more tasks.

“Based On.” As used herein, this term is used to describe one or more factors that affect a determination. This term does not foreclose additional factors that may affect a determination. That is, a determination may be solely based on those factors or based, at least in part, on those factors. Consider the phrase “determine A based on B.” While B may be a factor that affects the determination of A, such a phrase does not foreclose the determination of A from also being based on C. In other instances, A may be determined based solely on B.

The terms “a”, “an” and “the” mean “one or more”, unless expressly specified otherwise.

All terms of exemplary language (e.g., including, without limitation, “such as”, “like”, “for example”, “for instance”, “similar to”, etc.) are not exclusive of any other, potentially, unrelated, types of examples; thus, implicitly mean “by way of example, and not limitation . . . ”, unless expressly specified otherwise.

Unless otherwise indicated, all numbers expressing conditions, concentrations, dimensions, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” and “consisting of” limits the scope of a claim to the specified elements or method steps, plus those that do not materially affect the basis and novel characteristic(s) of the claimed subject matter (see Norian Corp. v Stryker Corp., 363 F.3d 1321, 1331-32, 70 USPQ2d 1508, Fed. Cir. 2004). Moreover, for any claim of the present invention which claims an embodiment “consisting essentially of” or “consisting of” a certain set of elements of any herein described embodiment it shall be understood as obvious by those skilled in the art that the present invention also covers all possible varying scope variants of any described embodiment(s) that are each exclusively (i.e., “consisting essentially of”) functional subsets or functional combination thereof such that each of these plurality of exclusive varying scope variants each consists essentially of any functional subset(s) and/or functional combination(s) of any set of elements of any described embodiment(s) to the exclusion of any others not set forth therein. That is, it is contemplated that it will be obvious to those skilled how to create a multiplicity of alternate embodiments of the present invention that simply consisting essentially of a certain functional combination of elements of any described embodiment(s) to the exclusion of any others not set forth therein, and the invention thus covers all such exclusive embodiments as if they were each described herein.

With respect to the terms “comprising,” “consisting of,” and “consisting essentially of,” where one of these three terms is used herein, the disclosed and claimed subject matter may include the use of either of the other two terms. Thus in some embodiments not otherwise explicitly recited, any instance of “comprising” may be replaced by “consisting of” or, alternatively, by “consisting essentially of”, and thus, for the purposes of claim support and construction for “consisting of” format claims, such replacements operate to create yet other alternative embodiments “consisting essentially of” only the elements recited in the original “comprising” embodiment to the exclusion of all other elements.

Moreover, any claim limitation phrased in functional limitation terms covered by 35 USC § 112(6) (post AIA 112(f)) which has a preamble invoking the closed terms “consisting of,” or “consisting essentially of,” should be understood to mean that the corresponding structure(s) disclosed herein define the exact metes and bounds of what the so claimed invention embodiment(s) consists of, or consisting essentially of, to the exclusion of any other elements which do not materially affect the intended purpose of the so claimed embodiment(s).

Devices or system modules that are in at least general communication with each other need not be in continuous communication with each other, unless expressly specified otherwise. In addition, devices or system modules that are in at least general communication with each other may communicate directly or indirectly through one or more intermediaries. Moreover, it is understood that any system components described or named in any embodiment or claimed herein may be grouped or sub-grouped (and accordingly implicitly renamed) in any combination or sub-combination as those skilled in the art can imagine as suitable for the particular application, and still be within the scope and spirit of the claimed embodiments of the present invention. For an example of what this means, if the invention was a controller of a motor and a valve and the embodiments and claims articulated those components as being separately grouped and connected, applying the foregoing would mean that such an invention and claims would also implicitly cover the valve being grouped inside the motor and the controller being a remote controller with no direct physical connection to the motor or internalized valve, as such the claimed invention is contemplated to cover all ways of grouping and/or adding of intermediate components or systems that still substantially achieve the intended result of the invention.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

A “computer” may refer to one or more apparatus and/or one or more systems that are capable of accepting a structured input, processing the structured input according to prescribed rules, and producing results of the processing as output. Examples of a computer may include: a computer; a stationary and/or portable computer; a computer having a single processor, multiple processors, or multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer (PC); a personal digital assistant (PDA); a portable telephone; application-specific hardware to emulate a computer and/or software, such as, for example, a digital signal processor (DSP), a field-programmable gate array (FPGA), an application specific integrated circuit (ASIC), an application specific instruction-set processor (ASIP), a chip, chips, a system on a chip, or a chip set; a data acquisition device; an optical computer; a quantum computer; a biological computer; and generally, an apparatus that may accept data, process data according to one or more stored software programs, generate results, and typically include input, output, storage, arithmetic, logic, and control units.

Those of skill in the art will appreciate that where appropriate, some embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Where appropriate, embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

“Software” may refer to prescribed rules to operate a computer. Examples of software may include: code segments in one or more computer-readable languages; graphical and or/textual instructions; applets; pre-compiled code; interpreted code; compiled code; and computer programs.

While embodiments herein may be discussed in terms of a processor having a certain number of bit instructions/data, those skilled in the art will know others that may be suitable such as 16 bits, 32 bits, 64 bits, 128s or 256-bit processors or processing, which can usually alternatively be used. Where a specified logical sense is used, the opposite logical sense is also intended to be encompassed.

The example embodiments described herein can be implemented in an operating environment comprising computer-executable instructions (e.g., software) installed on a computer, in hardware, or in a combination of software and hardware. The computer-executable instructions can be written in a computer programming language or can be embodied in firmware logic. If written in a programming language conforming to a recognized standard, such instructions can be executed on a variety of hardware platforms and for interfaces to a variety of operating systems. Although not limited thereto, computer software program code for carrying out operations for aspects of the present invention can be written in any combination of one or more suitable programming languages, including an object oriented programming languages and/or conventional procedural programming languages, and/or programming languages such as, for example, Hypertext Markup Language (HTML), Dynamic HTML, Extensible Markup Language (XML), Extensible Stylesheet Language (XSL), Document Style Semantics and Specification Language (DSSSL), Cascading Style Sheets (CSS), Synchronized Multimedia Integration Language (SMIL), Wireless Markup Language (WML), Java™, Jini™, C, C++, Smalltalk, Perl, UNIX Shell, Visual Basic or Visual Basic Script, Virtual Reality Markup Language (VRML), ColdFusion™ or other compilers, assemblers, interpreters or other computer languages or platforms.

Computer program code for carrying out operations for aspects of the present invention may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

A network is a collection of links and nodes (e.g., multiple computers and/or other devices connected together) arranged so that information may be passed from one part of the network to another over multiple links and through various nodes. Examples of networks include the Internet, the public switched telephone network, the global Telex network, computer networks (e.g., an intranet, an extranet, a local-area network, or a wide-area network), wired networks, and wireless networks.

The Internet is a worldwide network of computers and computer networks arranged to allow the easy and robust exchange of information between computer users. Hundreds of millions of people around the world have access to computers connected to the Internet via Internet Service Providers (ISPs). Content providers (e.g., website owners or operators) place multimedia information (e.g., text, graphics, audio, video, animation, and other forms of data) at specific locations on the Internet referred to as webpages. Websites comprise a collection of connected, or otherwise related, webpages. The combination of all the websites and their corresponding webpages on the Internet is generally known as the World Wide Web (WWW) or simply the Web.

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

The flowchart and block diagrams in the figures illustrate the architecture, functionality, and operation of possible implementations of systems, methods and computer program products according to various embodiments. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

Further, although process steps, method steps, algorithms or the like may be described in a sequential order, such processes, methods and algorithms may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

It will be readily apparent that the various methods and algorithms described herein may be implemented by, e.g., appropriately programmed general purpose computers and computing devices. Typically, a processor (e.g., a microprocessor) will receive instructions from a memory or like device, and execute those instructions, thereby performing a process defined by those instructions. Further, programs that implement such methods and algorithms may be stored and transmitted using a variety of known media.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article.

The functionality and/or the features of a device may be alternatively embodied by one or more other devices which are not explicitly described as having such functionality/features. Thus, other embodiments of the present invention need not include the device itself.

The term “computer-readable medium” as used herein refers to any medium that participates in providing data (e.g., instructions) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random-access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, removable media, flash memory, a “memory stick”, any other memory chip or cartridge, a carrier wave as described hereinafter, or any other medium from which a computer can read.

Various forms of computer readable media may be involved in carrying sequences of instructions to a processor. For example, sequences of instruction (i) may be delivered from RAM to a processor, (ii) may be carried over a wireless transmission medium, and/or (iii) may be formatted according to numerous formats, standards or protocols, such as Bluetooth, TDMA, CDMA, 3G.

Where databases are described, it will be understood by one of ordinary skill in the art that (i) alternative database structures to those described may be readily employed, (ii) other memory structures besides databases may be readily employed. Any schematic illustrations and accompanying descriptions of any sample databases presented herein are exemplary arrangements for stored representations of information. Any number of other arrangements may be employed besides those suggested by the tables shown. Similarly, any illustrated entries of the databases represent exemplary information only; those skilled in the art will understand that the number and content of the entries can be different from those illustrated herein. Further, despite any depiction of the databases as tables, an object-based model could be used to store and manipulate the data types of the present invention and likewise, object methods or behaviors can be used to implement the processes of the present invention.

A “computer system” may refer to a system having one or more computers, where each computer may include a computer-readable medium embodying software to operate the computer or one or more of its components. Examples of a computer system may include: a distributed computer system for processing information via computer systems linked by a network; two or more computer systems connected together via a network for transmitting and/or receiving information between the computer systems; a computer system including two or more processors within a single computer; and one or more apparatuses and/or one or more systems that may accept data, may process data in accordance with one or more stored software programs, may generate results, and typically may include input, output, storage, arithmetic, logic, and control units.

A “network” may refer to a number of computers and associated devices that may be connected by communication facilities. A network may involve permanent connections such as cables or temporary connections such as those made through telephone or other communication links. A network may further include hard-wired connections (e.g., coaxial cable, twisted pair, optical fiber, waveguides, etc.) and/or wireless connections (e.g., radio frequency waveforms, free-space optical waveforms, acoustic waveforms, etc.). Examples of a network may include: an internet, such as the Internet; an intranet; a local area network (LAN); a wide area network (WAN); and a combination of networks, such as an internet and an intranet.

As used herein, the “client-side” application should be broadly construed to refer to an application, a page associated with that application, or some other resource or function invoked by a client-side request to the application. A “browser” as used herein is not intended to refer to any specific browser (e.g., Internet Explorer, Safari, FireFox, or the like), but should be broadly construed to refer to any client-side rendering engine that can access and display Internet-accessible resources. A “rich” client typically refers to a non-HTTP based client-side application, such as an SSH or CFIS client. Further, while typically the client-server interactions occur using HTTP, this is not a limitation either. The client server interaction may be formatted to conform to the Simple Object Access Protocol (SOAP) and travel over HTTP (over the public Internet), FTP, or any other reliable transport mechanism (such as IBM® MQSeries® technologies and CORBA, for transport over an enterprise intranet) may be used. Any application or functionality described herein may be implemented as native code, by providing hooks into another application, by facilitating use of the mechanism as a plug-in, by linking to the mechanism, and the like.

Exemplary networks may operate with any of a number of protocols, such as Internet protocol (IP), asynchronous transfer mode (ATM), and/or synchronous optical network (SONET), user datagram protocol (UDP), IEEE 802.x, etc.

Embodiments of the present invention may include apparatuses for performing the operations disclosed herein. An apparatus may be specially constructed for the desired purposes, or it may comprise a general-purpose device selectively activated or reconfigured by a program stored in the device.

Embodiments of the invention may also be implemented in one or a combination of hardware, firmware, and software. They may be implemented as instructions stored on a machine-readable medium, which may be read and executed by a computing platform to perform the operations described herein.

More specifically, as will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

In the following description and claims, the terms “computer program medium” and “computer readable medium” may be used to generally refer to media such as, but not limited to, removable storage drives, a hard disk installed in hard disk drive, and the like. These computer program products may provide software to a computer system. Embodiments of the invention may be directed to such computer program products.

An algorithm is here, and generally, considered to be a self-consistent sequence of acts or operations leading to a desired result. These include physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers or the like. It should be understood, however, that all of these and similar terms are to be associated with the appropriate physical quantities and are merely convenient labels applied to these quantities.

Unless specifically stated otherwise, and as may be apparent from the following description and claims, it should be appreciated that throughout the specification descriptions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

Additionally, the phrase “configured to” or “operable for” can include generic structure (e.g., generic circuitry) that is manipulated by software and/or firmware (e.g., an FPGA or a general-purpose processor executing software) to operate in a manner that is capable of performing the task(s) at issue. “Configured to” may also include adapting a manufacturing process (e.g., a semiconductor fabrication facility) to fabricate devices (e.g., integrated circuits) that are adapted to implement or perform one or more tasks.

In a similar manner, the term “processor” may refer to any device or portion of a device that processes electronic data from registers and/or memory to transform that electronic data into other electronic data that may be stored in registers and/or memory. A “computing platform” may comprise one or more processors.

Embodiments within the scope of the present disclosure may also include tangible and/or non-transitory computer-readable storage media for carrying or having computer-executable instructions or data structures stored thereon. Such non-transitory computer-readable storage media can be any available media that can be accessed by a general purpose or special purpose computer, including the functional design of any special purpose processor as discussed above. By way of example, and not limitation, such non-transitory computer-readable media can include RAM, ROM, EEPROM, CD-ROM or other optical disk storage, magnetic disk storage or other magnetic storage devices, or any other medium which can be used to carry or store desired program code means in the form of computer-executable instructions, data structures, or processor chip design. When information is transferred or provided over a network or another communications connection (either hardwired, wireless, or combination thereof) to a computer, the computer properly views the connection as a computer-readable medium. Thus, any such connection is properly termed a computer-readable medium. Combinations of the above should also be included within the scope of the computer-readable media.

While a non-transitory computer readable medium includes, but is not limited to, a hard drive, compact disc, flash memory, volatile memory, random access memory, magnetic memory, optical memory, semiconductor based memory, phase change memory, optical memory, periodically refreshed memory, and the like; the non-transitory computer readable medium, however, does not include a pure transitory signal per se; i.e., where the medium itself is transitory.

Challenges and inefficiencies characteristic of traditional HMOs may include labyrinthine billing, insurance, and new patient intake procedures. When coupled with exceedingly complex therapies and treatment requirements, such HMOs may be difficult for customers and/or patients to participate in, leaving such persons often seeking realistic and reliable cost-effective alternatives. Further, such HMOs may not uniformly implement or even properly consider advanced computer-science based data analytic techniques, inclusive of artificial intelligence (“AI”), machine learning, neural networks, image recognition, blockchain-based technologies, AI-based virtual health assistants, data management platforms (DMPs), genomics, and/or the like for the purposes of streamlining existing processes to achieve cost efficiencies.

The system herein transforms a highly fragmented, hospital-centered healthcare delivery system into a patient-centered single universally federated global healthcare system by providing high-quality care and delivering revolutionary smart digital solutions of micro services software, processes, and medical devices to patients at affordable or reduced costs. The system enables digital transformation technologies embracing artificial intelligence and machine learning for innovative algorithms, third-party data for insights and outcomes, bionics and biometrics for cryptographically secure Protected Health Information (PHI) and Personally Identifiable Information (PII), and blockchain technologies to secure distributed ledger technologies (DLT) transactions. Its world-wide multi-region cloud infrastructures provide secure web service and smartphone portals where providers and device manufacturers are given incentives while patients, participants, and other users gain digital currency tokens to pay for their healthcare costs at a reduced price. The platform will continuously and independently test; adapt and implement healthcare devices, processes, software tools, as well as newer digital emerging technologies that are known forefronts for healthcare delivery.

The system, known as “A-Medicare”, transforms and future-proofs many processes, services, and businesses by adopting newer digital technology, singular and blockchain databases, machine learning, third-party data, patient data analytics, and artificial intelligence to gain accurate insight and outcomes into drug usage, diagnosis, healthcare, and patient health lifestyles.

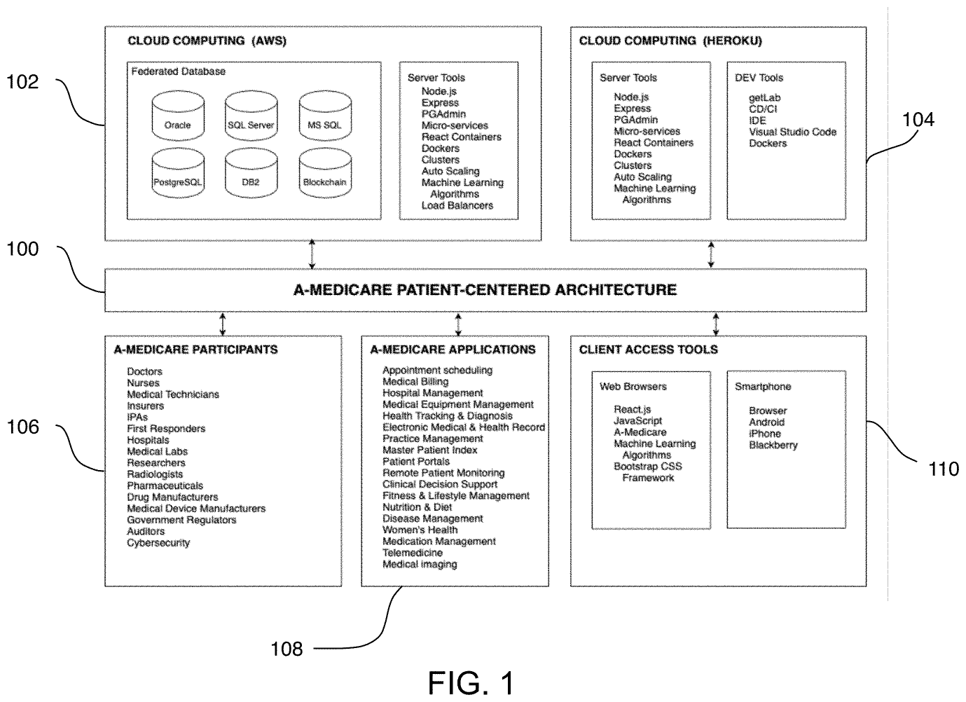

FIG. 1 illustrates a schematic view of the interaction of the A-Medicare Patient-Centered Architecture 100, including integration with Cloud Computing (AWS) 102, Cloud Computing (HEROKU) 104, Participants 106, A-Medicare Applications 108, and Client Access Tools 110. Because the system emulates functionality of the entire healthcare system, the A-Medicate Participants 106 include, but are not limited to, doctors, nurses, medical technicians, insurers, IPAs, first responders, hospitals, medical labs, researchers, radiologists, pharmaceutical companies, drug manufactures, medical device manufacturers, government regulators, auditors, and cybersecurity experts. Each participant category has an individualized portal where relevant information is displayed. Two-way interactions are also performed in each individual's portal. However, some participants may have passive participation, such as a database, where information is simply disseminated when queried from an application of the system.

A-Medicate Applications 108 provide an full suite of services directed to the healthcare industry, and include, but are not limited to, appointment scheduling, medical billing, hospital management, medical equipment management, health tracking and diagnosis, electronic medical and health records, practice management, a master patient index, patient portals, remote patient monitoring, clinical decision support, fitness and lifestyle management, nutrition and diet services, disease management, women's health, medication management, telemedicine, medical imagine, and reward-based programs, such as advertisements.

Client access tools 110 may include, but are not limited to, web browsers and smart phones. However, the system may also integrate with other technologies, including professional medical devices, but may also integrate with personal electronics and Internet of Things devices, such as wearable technology, voice assistant technology, and robotic assistants.

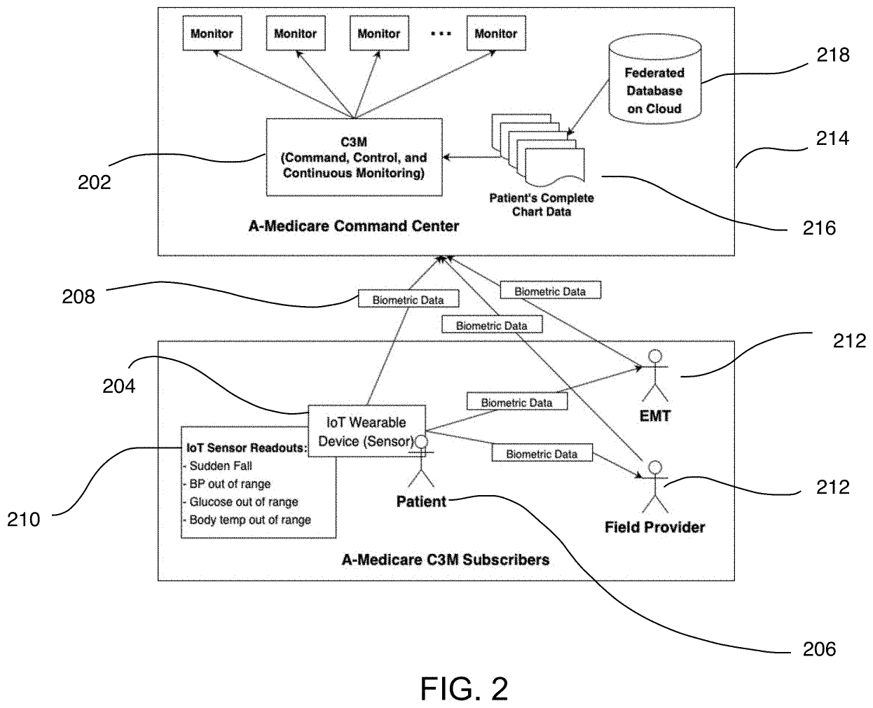

FIG. 2 illustrates an example “cloud”-based system and the interaction of a user's wearable device on the system. The software and hardware infrastructure performs command, control, and continuous monitoring (C3M) 202 of connected network of client hospital operations and use Internet of Things (IoT) and bionic devices 204 worn by patients 206 to track and analyze users' health data 208. The C3M 202 obtains and processes real-time data 216 of patients 206 such as location, chart, doctor, provider, and medical insurance when first symptoms appear. The A-Medicare Command Center 214 includes a federated cloud database 218, which interacts with patients data 216 that is continuously monitored by the C3M 202. Real-time changes cause alerts to be pushed to medical staff and contracted medical professionals 212. Further, wearable devices 204 include a plurality of sensors 210 which sense for sudden falls, blood pressure, glucose levels, and body temperature.

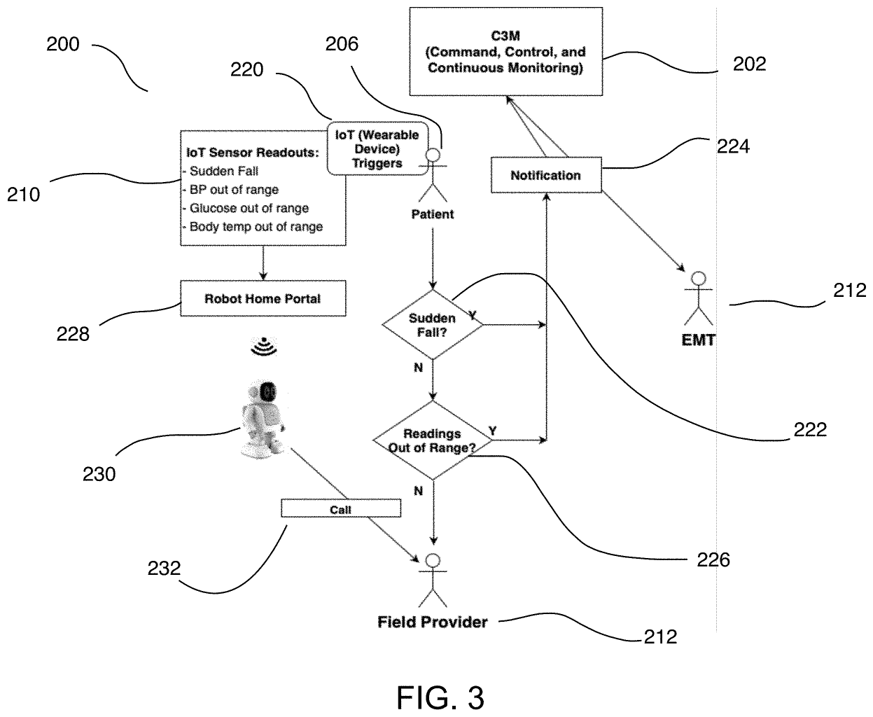

FIG. 3 is an exemplary flow diagram of the notification system 200 involving a wearable 204. A patient 206 is shown to have a wearing a wearable device 204 with triggers 220. The system 200 detects whether a fall has occurred 222. If a sudden fall 222 has occurred, the system sends a notification 224 to the C3M 202, which in turn notifies a medical professional 212. If a sudden fall 222 has not been detected, the system queries whether the readings are out of range 226. If the readings are out of range 226, then the system 200 sends a notification 224 to the C3M, which notifies medical professionals 212. If the readings are not out of range, a medical professional 212, such as a field provider is contacted. In some embodiments, the IoT sensor readouts, 210 will also integrate with a home portal 228, which may direct a robotic assistant 230 to check in, and, if needed, contact a medical professional 212.

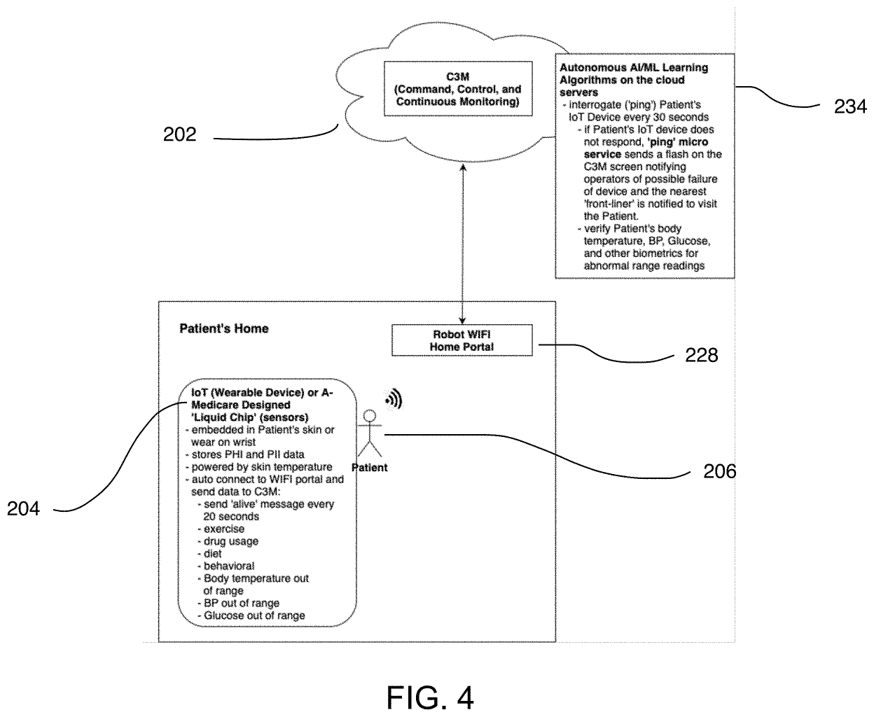

In order to provide constant monitoring of a patient's health conditions, the Internet of Things (IoT) devices 204 are implemented to provide constant feedback which thereby influences different executable tasks in the system. These IoT device 204 can be wearables (similar to Fitbit or Apple Watch), and bionic nano devices such as liquid chip that is embedded into a patient skin whose electrical power is harvested from the skin temperature. These devices 204 securely store selected patient ‘diary’ data such as PII and PHI. Once the nano or IoT device 204 is near the vicinity of a secure Wi-Fi network, it will auto connect, and new data are uploaded into a central database, which in FIG. 4, as an illustration of a patient's IoT device and autonomous server communications. The central database 218 is continuously updated by the patient's daily activities such as exercise, drug, or medication usage, diet, and other behavioral data 208. Use of the bionic and wearable devices 204, employ smart software tools and a notification protocol 224 that are linked to the data warehouse, third-party data, and web intelligence to identify potential failures of the devices (IoT) and changes in the patient's medical data long before a symptom occurs.

Autonomous AI/ML algorithms 234 are also applied on the cloud servers, such as the C3M 202. These algorithms ping patient's IoT devices 204 every 30 seconds. If the patient's device does not respond, the system pings a microservice and sends a flash on the C3M screen notifying operators of possible failure of the device and the nearest front-line worker is notified to visit the patient. The algorithms verify the patient's body temperature, blood pressure, glucose, and other biometric data for abnormal readings and relays this information to the C3M and on to the healthcare worker.

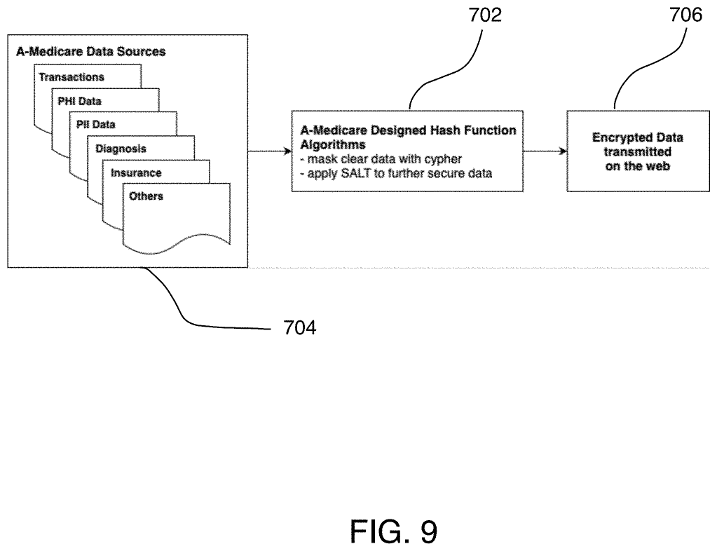

Cybersecurity measures are implemented using blockchains, immutable ledgers, and hash techniques on selected Personally Identifiable Information (PII), Protected Health Information (PHI), diagnosis, and DNA to comply with HIPAA and other governing bodies, as shown in FIG. 9 and discussed below.

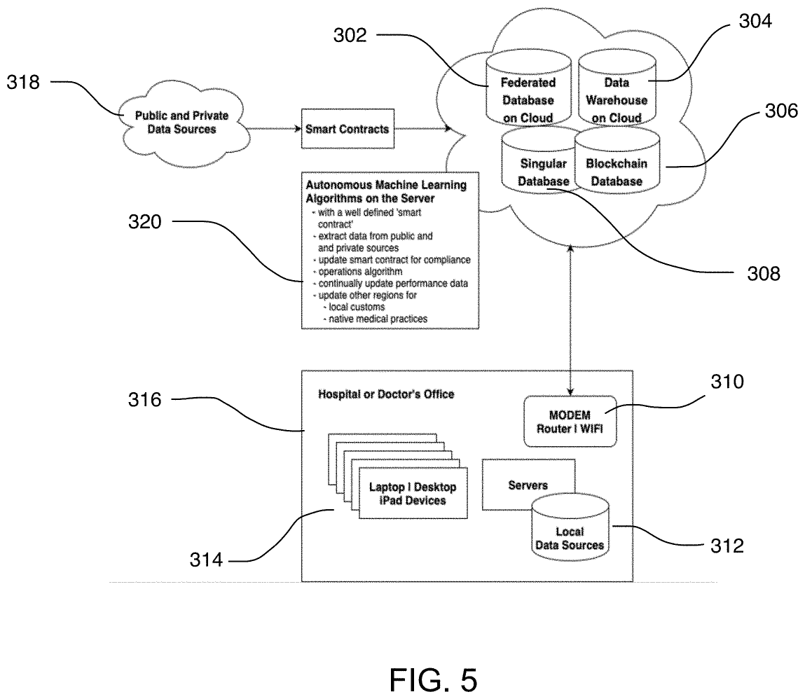

Exemplary embodiments implement a federated database 302, as may be seen in FIG. 5. The federated database 302 may further consist of localized databases 312, or connected through a internet gateway 310 to public and private databases 318, blockchain databases 306, cloud-based data warehouses 304, and a single centralized enterprise database 308 for the A-Medicare system. The federated database 302 information is integrated by using machine learning algorithm 320, operations, and processes in geographical regions for performance, ethical practices, local customs, native medical practices, and more granular patient data. These data and insights are presented to doctors and decision makers, such as in the doctor's office 316, on a large display's panels or personal devices 314, such as laptops, desktops with a graphic user interface, or with a tablet, such as iPad or other tablet type devices.

The system establishes a global alliance with insurance companies, service providers, pharmaceuticals, device manufacturers, financial institutions, insurers, governments, and aid organizations to establish policies and standards in the ever-growing complexities in the healthcare practice worldwide. This allows the system to cross-connect over a broad range of technologies to form one synergistic platform which, in some embodiments, allows inputs to be transformed in to a universal protocol to be processed by the system from the raw data associated with each of the with insurance companies, service providers, pharmaceuticals, device manufacturers, financial institutions, insurers, governments, and aid organizations.

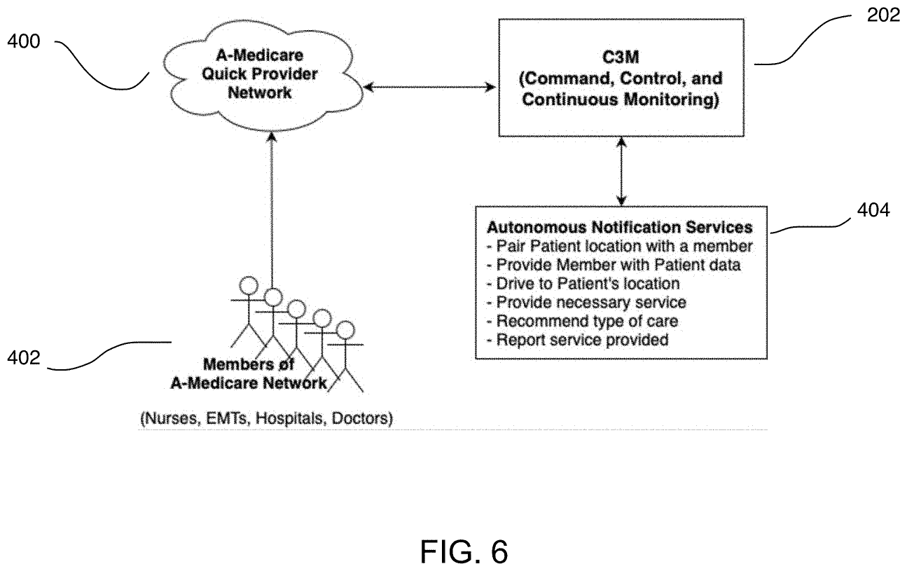

The system creates a network 400 of ‘quick-providers’ 402 composed of front liners such as nurses, doctors, emergency medical technicians (EMT) who are in close proximity of the patient's household. FIG. 6 illustrates an exemplary embodiment of the A-Medicare Quick-Provider Network. The ‘quick-providers’ 402 can provide immediate care of the patient during emergencies when the nearest hospital, medical clinic, or healthcare provider facility is miles and hours away. The system records the geolocation of each user device, including both patient and service provider, and allows the system to match nearby service providers with patients while filtering out providers that may be assisting other patients or who would not meet the required response time. This also removes obstacles with immediate dispatch of service providers, because the system monitors the locations of the service providers in real-time, which cannot be accomplished by a dispatcher since multiple geolocations may be constantly moving and the closest service provider could change by the second.

The C3M 202 receives the A-Medicare Quick-Provider Network data and processes the information for an Autonomous Notification System 404. The Autonomous Notification System 404 can pair patient locations with a member of the A-Medicare network 402. It can also Provide the member 402 with patient data, driving directions, and suggest necessary services, recommend types of care, and report services provided.

In addition to using geolocation for matching service providers, the system also incorporates an early warning and tracking system to monitor organ donors worldwide and document logistics for quick and safe preparation, transportation, and transplant to a patient who is on the top priority list to receive the organ.

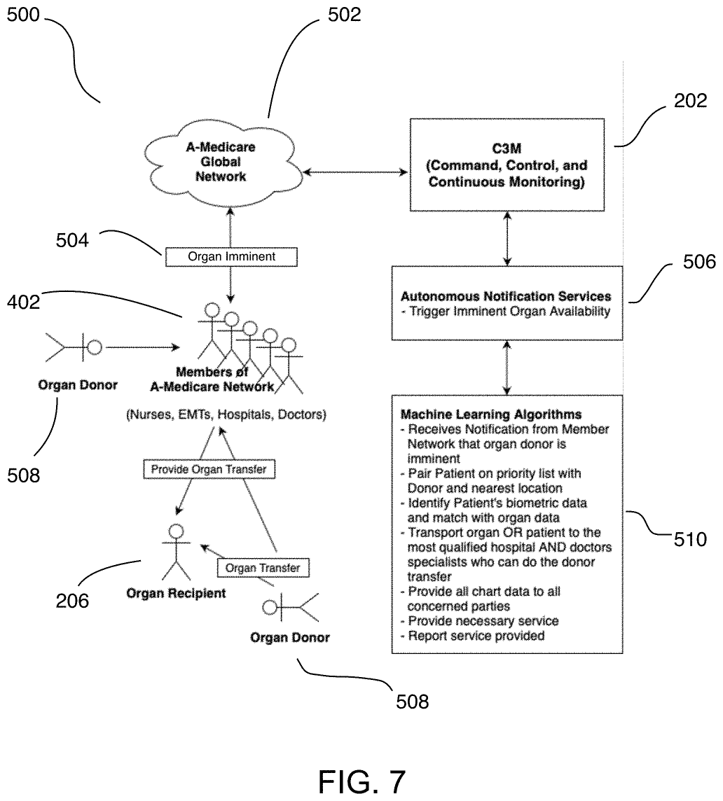

Some implementations employ an early warning tracking system 500, as illustrated in FIG. 7, to monitor organ donors. The A-Medicare Global Network 502 communicates bi-directionally with the C3M 202. The A-Medicare Global Network 502 monitors for imminent availability or an organ 504. When an organ becomes available from an organ donor 508, an Autonomous Notification Service 506 triggers a notification that is pushed to relevant parties, including members of the A-Medicare Network 402 and the organ recipient, the patient 206. The Autonomous Notification Service 506 relies on a machine learning algorithm 510, which processes which recipient will be matched to receive the organ by receiving the notification from a member network that an organ donation is imminent, pairing the patient on a priority list with the donor and nearest location, identifying the patient's biometric data and matching with the organ data, processing and coordinating transportation of the organ and/or patient to the most qualified hospital and doctor specialist who can execute the donor transfer, providing all chart data to all involved parties, coordinating the necessary services, and reporting the services provided. The machine learning algorithm may also amend recommended treatments based on the potential for a donor match or whether no match is imminent, and other methods of treatment are then recommended and/or applied.

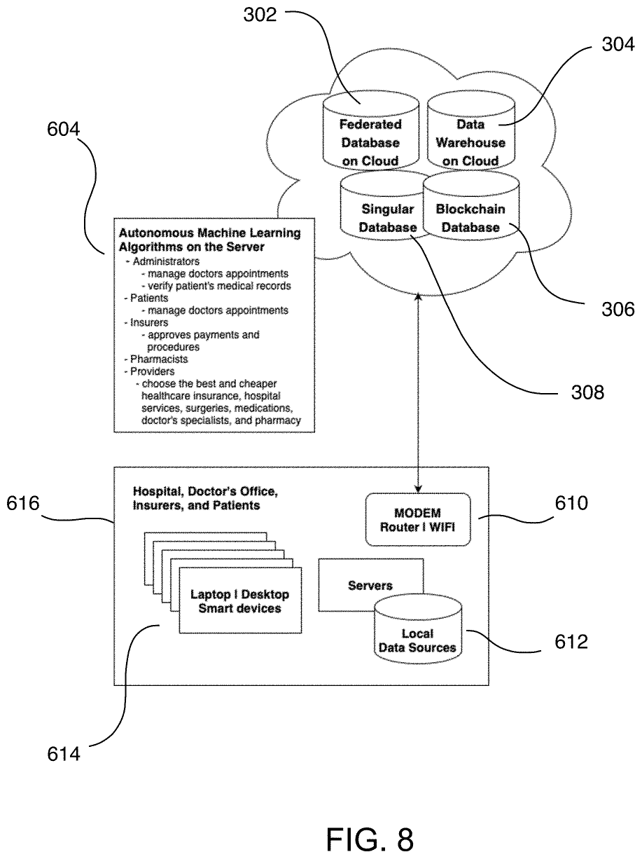

In some exemplary embodiments, as illustrated in FIG. 8, a laptop, desktop, or smart device 602, such as a smart phone or tablet, includes a Client Web Browser and Smartphone App includes the client master ‘engine’ developed around a patient-centered interface using a combination of server-, browser-, and machine learning-based algorithmic software 604 using Python, SQL, node.js, react.js, and the Smartphone device's native operating system such as Android and Apple iOS. Providers, patients, insurers, pharmacists, administrators and other users will have access to a secure browser-based web client and SmartPhone native apps implementing A-Medicare's unique artificial intelligence-based architecture and services.

In an exemplary embodiment of the system showing the interaction of the system with the Hospital, Doctor's Office, Insurers, and Patient's interfaces 616, illustrated in FIG. 8, information is pulled from partner databases, including federated databases 302 on cloud storage, data warehouses 304 on cloud storage, singular databases 308, and blockchain databases 306. The information is communicated through a network to a user's modem, router, or other network connection 610. It is then stored on local data sources 612, and displayed on the user's device 614.

The system includes a rule-based panel system. By signing into the strategically designed rule-based panel system, a patient or administrator can manage doctor's appointments, verify the patient's medical records and providers, choose the best and cheaper health care insurance, hospital services, surgeries, doctors' visits, medications, doctor specialist, and pharmacy.

FIG. 9 illustrates encryption of data within the system. Using custom cryptographic hash algorithms 702, the client engine encrypts sensitive transactional data 704 such as insurance, diagnosis, financial, Personally Identifiable Information (PII) and Protected Health Information (PHI) as it is used by the A-Medicare web service in the various page navigation by the user. This data 704 is transmitted as encrypted data for transmission on the web 706. The client engine randomly updates the encryption keys at various times to prevent Hackers from knowing the exact key in those random times.

Depending on how the user navigates or use the A-Medicare web service or Smartphone app, hundreds of autonomous micro services (like ‘daemons’ in Unix) or micro robots (bots) continuously work in the background doing household tasks such as searching or detecting unusual cybersecurity activity like ransomware, DDoS, and phishing software about to ‘knock’ off, kill or hijack windows client services or inject itself in the various services of the host operating system and other application software of the device.

The system embeds cognitive technology into the client ‘engine’ as the user navigates various pages of the A-Medicare web service applications or Smartphone app in concert with the server-based services and processes. The client ‘engine’ will detect and collect context sensitive words and phrases on the current page of the web or app into a local cache then destroys or updates this cache at random when specific tasks of the web service is completed.

At user's pre-agreed consent (via a pop or dialog box) or smart contract, the A-Medicare platform collects information into a singular data warehouse using locally developed cryptographic and hash techniques. This data can be collected and presented for user insights, audits, and outcomes in large display or iPad type devices for further analysis by doctors and decision makers. The system uses custom hashing and cryptographic techniques, securely querying singular and blockchain validation database clusters based on thousands of pre-created scripts and micro services that are machine learning-based, well documented and easy to use for any type of users.

For performance, database queries are redirected by the Federated master agent against the partitioned singular and blockchain database regional location (as an example Sydney) where the web service client or Smartphone device being used based on its location.

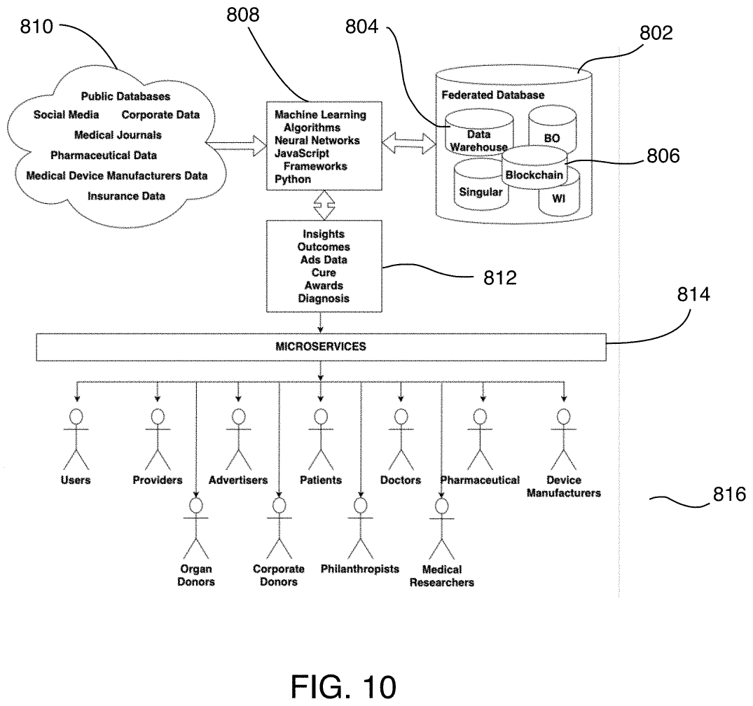

In some exemplary implementations, as may be appreciated in FIG. 10, the system incorporates Federated Database and Blockchain technologies. The A-Medicare platform will be composed of several federated databases 802, data warehouses 804, and blockchain databases 806 that may be built or populated by health data such as epidemiological research, disease and bio hazards surveillance and other data from various sources including local data, PII, PHI, diagnostic, pharmaceutical, medical and drug research, peer-to-peer, insight, chart, patient, user profile, meta data, and data automatically sent by nano and IoT devices. This data is synchronized cross-platform in real-time. For example, Pseudo Sample Code to synchronize data from CDC:

```
INSERT INTO<schema>.<database-table>values (‘...
  <data to be imported into A-Medicare federated database> ...′
  <disease-type> <-- encrypt-with-hash)
FROM <CDC-database-disease-information>
WHERE <infectious-data> are <current-date> minus 60 days
```

These databases will be physically hosted in various regions around the globe in a highly secure cloud infrastructure using A-Medicare's add-on layer of cybersecurity that's virtually impossible to hack.

Blockchain databases capture general ledger data as another technique in hiding PII, PHI, diagnostic data, financial, and other healthcare data.

An example of Pseudo Same Data Elements to Generate the Genesis Block and Succeeding Transactions are as follows:

```
const GENESIS_DATA = {
  timestamp: Date.now( ),
  lastHash: ′last-hash-can-be-anything′,
  hash: ′can-be-anything′,
  difficulty: 3,
  nonce: 0,
  data: [′This is the genesis block.′]
};
Then used in Blockchain class. When creating an instance of the
Blockchain class a method genesis( ) from Block class is called in
the constructor to create the genesis block.
class Blockchain {
  constructor( ) {
    this.chain = [Block.genesis( )];
  }
...
}
class Block {
constructor({ timestamp, lastHash, hash,
data, nonce, difficulty }) {
// nonce & difficulty: for PoW system to account how
// new blocks could be created
    this.timestamp = timestamp;
    this.lastHash = lastHash;
    this.hash = hash;
    this.data = data;
    this.nonce = nonce;
    this.difficulty = difficulty;
  }
  static genesis( ) {
    return new this(GENESIS_DATA);
  }
...
}
```

FIG. 10 also illustrates the system architecture, wherein the machine learning algorithms, neural networks, and other programming frameworks 808 pull information from the federated databases 802 and data from established sources 810, including public databases, social media, corporate data, medical journals, pharmaceutical data, medical device manufacturers data, and insurance data, which in turn gets processed in accordance with a module 812 for processing insights, outcomes, ad data, cures, awards, and diagnosis that is then sent to the appropriate microservices 814, whereby the outcome is communicated to the appropriate party 816, including users, providers, advertisers, patients, doctors, pharmaceutical industry workers, device manufacturers, organ donors, corporate donors, philanthropists, and medical researchers.

In some implementations, the A-Medicare system provides real-time decision support system composed of thousands of queries and services based on machine learning algorithms displaying them into various large displays and iPad/tablet type devices. The machine learning algorithm automatically learns and improves the processes from experience without being explicitly programmed or manually feed other data.

The iPad/tablet devices are utilized by doctors and nurses for C3M type monitoring and quick response to provide service. These display devices show patient charts, patient-doctor-nurse interaction, dosing patterns, drug usage, patient surveillance, upcoming tests, diagnostic data, genetic testing, personalized digital health plans, and other healthcare information.

The system provides information on self-treatment of diseases and improves communications between patients and care professionals so they can manage patient's health at home. Capturing the process via remote monitoring and analyzing the results will improve home-health administration of drugs and appropriated care.

In many embodiments, medical records, diagnosis data, and charts are protected at a high level. The A-Medicare system implements Confidentiality, Integrity, and Availability, known as the cybersecurity “CIA” triad. CIA protects patient's PII, PHI, diagnostics, insurance, and financial data and make this data available when needed.

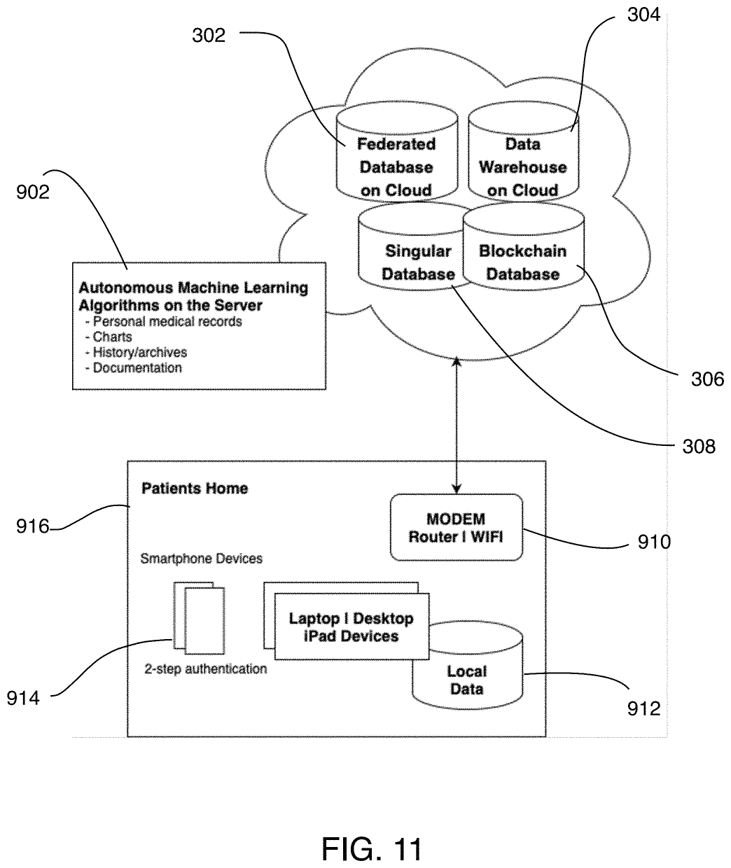

FIG. 11 illustrates an exemplary embodiment of the system. The system shows federated databases 302, warehouse databases 304, singular databases 308, and blockchain databases 306, wherein autonomous machine learning algorithms 902 are integrated to the server. The algorithm 902 is configured to provide and update personal medical records, charts, history and archives, and documentation. The data from this algorithm 902 is received by the user interface of a patient's home 916 through a network connection 910 and stored as local data 912. The information is then displayed on a user's device 914, which may be a smart phone in some embodiments.

Personal medical records, charts, history archives, and documentation of a doctor's visit with a patient are kept securely in singular 308 and blockchain 306 databases both at-rest and in-transit using machine learning algorithms 902. This confidential data can only be accessed with 2-step authentication on top of advanced encryption techniques and cybersecurity policy and other government mandated policy such as HIPAA to protect the patients PHI and PII. Further, before clear data is saved into the applicable database it is masked with an A-Medicare machine learning designed algorithm. Translation code is similar to SALT in encryption to masked clear data This process will be virtually impossible to hijack or intercept while the data is on transit to its final destination. An exemplary Pseudo Sample Code to Add Additional Security is provided:

```
INSERT INTO <schema>.<database-table>values
  (‘... <data to be imported into A-Medicare federated database> ...′
  (replaced with) <translate-code>
FROM <source-database-with-clear-data>
WHERE ...
```

In seeking appointments with medical practitioners, this secure healthcare data can be accessed using authorization and 2-step authentication via browser-based and Smartphone app and role-base and machine learning algorithm(s).

Some embodiments of the invention include advertisement, web service, and smartphone app content to keep the user engaged and thereby encouraged to be informed on their health status, which is intended to facilitate healthier lifestyles. One of the ways the system does this, in particular, is through an advertisement supported reward system.

A-Medicare advertisement pop-up dialogs are triggered and presented to the web service and Smartphone clients according to the user's likes, postings, profiles, words, phrases, and also health information, and the device location. These insights or outcomes are a result of custom designed machine learning algorithms that learn accurate and deep intuitive understanding of the user, and as a result of extraction from a singular data warehouse, blockchain database, and other public records.

Using modern libraries such as react.js, the client application displays a custom advertisement pop-up dialog according to the user insights. Pop-up dialogs are designed to be less intrusive so it will not affect or block content that the user is reading. By clicking the advertisement pop-up dialog, A-Medicare awards portion of the advertisement revenue back to the user as A-Medicare cryptocurrency token in some embodiments. The number of seconds and minutes that the user spent and the type of content that the user is interested on the advertiser's corporate website will be collected and stored into the Data Warehouse for later insights.

Awarding users, A-Medicare cryptocurrency tokens encourages the user to search for more information provided free by the A-Medicare platform. Users' likes, comments, opinions, thoughts, postings, and suggestions will contribute to improving healthcare, pharmaceutical, medical products, and healthcare related services in the future. Further, the system motivates user to perform further research on information like self-management of minor diseases or ailments. Compensation received from interaction with the ads, whether it be a crypto token or another digital credit, may be pooled into a single account.

In addition to the user receiving compensation for viewing advertising content, a percentage of the ad revenue supports other aspects of the A-Medicate system. A-Medicare's ‘after the fact’ insurance type payments absorbs the cost and fund a patient's surgery procedure. A-Medicare initially funds the cost while getting funds from people like good-hearted subscribers, philanthropists, as well as users with surplus tokens wanting to share to patients. For user's that are considered good Samaritans for donating credits to others, those users are awarded with additional tokens.

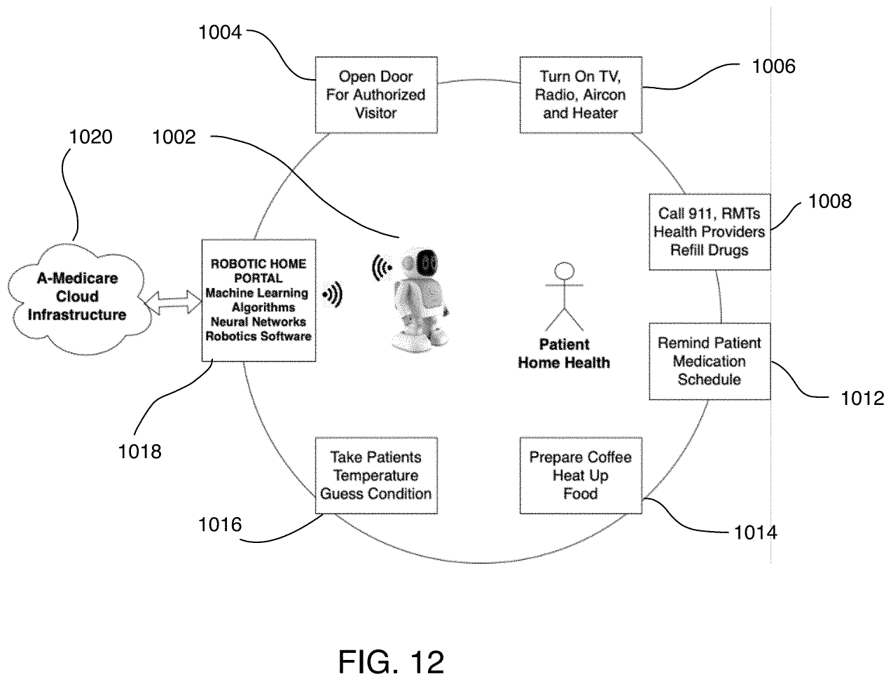

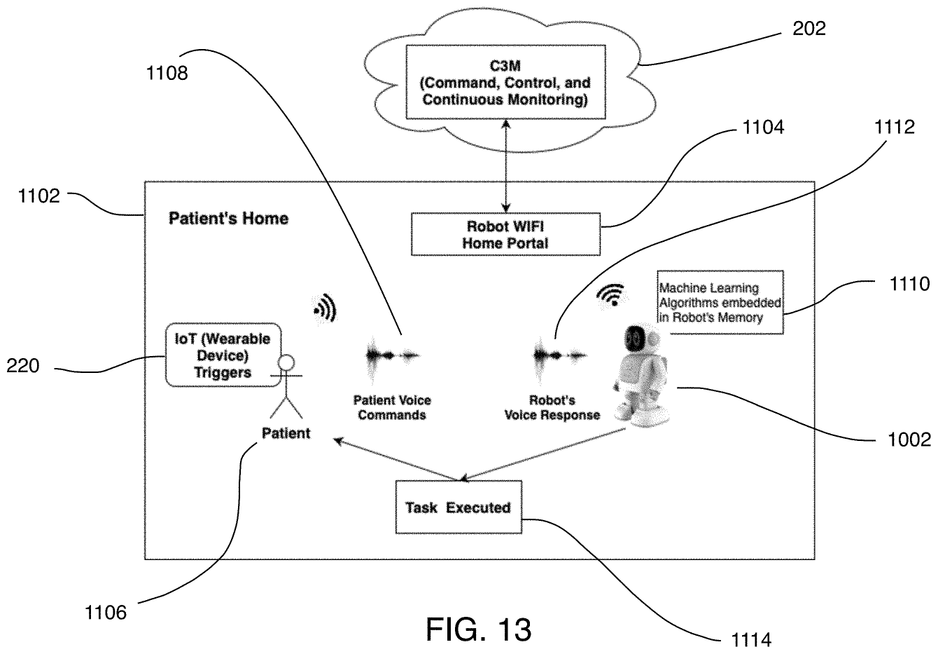

Some implementations involve integration with virtual assistant technologies including, but not limited to, voice assistants and robots, transforming medical records and diagnosis into a ‘Siri’ or ‘Alexa’-like voice assistant using blockchain and machine learning technology. The system may include robots, as shown in FIGS. 12 and 13, acting as Personal Health Assistants. Furthermore, A-Medicare may collaborate with third-party robot manufacturers whose robot meets rigid specifications.

The robots 1002 may be configured to understand English commands and responds accordingly to said commands, for example, opening the door 1004 for home healthcare service, turning on the television 1006 for news and other entertainment. These robots 1002 can also call 911, RMTs, healthcare providers, or refill drugs 1008. It can remind patient of medication schedule 1012, prepare certain beverages, get items from the refrigerator, heat up food, 1014 and administer other mundane tasks in service of his/her master, including taking temperature 1016. Embedded in the robot's 1002 memory are thousands of A-Medicare designed machine learning algorithm 1018 that automatically learn and improve from experience without being explicitly programmed of feed manually data or processes. The algorithms 1018 also communicate bi-directionally with the A-Medicare Cloud Infrastructure 1020.

FIG. 13 illustrates the patient and robot communications. Microphones with Bluetooth or WIFI connectivity are installed strategically throughout the patient's home 1102. All devices are automatically connected to a specialized Robotic Home Portal 1104 (much like a WIFI router). When service is needed Patient 1106 say commands 1108 in English directed to the Healthcare Robot 1002. Robot 1002 hears the ‘raw’ command 1108 if within hearing range or received via Robotic Home Portal's 1104 WIFI. The Robot 1002 receives the command 1108, interrogate its CPU (computer central processing) memory and compare the English commands 1108 from thousands of AI (Artificial Intelligence) based Machine Learning processes 1110 both inside the Robot 1002 and the A-Medicare Federated database and C3M 202. The Robot interprets the command 1108, responds 1112 to the Patient 1106 using voice synthesis through its own speakers. The Robot executes actionable tasks 1114 as requested by the Patient. In some embodiments, the user 1106 also wears a wearable device, with triggers 220, that send signals to the robot 1002 without the use of voice commands.

All commands 1108 from the Patient 1106 heard by the Robot 1002, its responses 1112, and tasks executed 1114, comes from Machine Learning algorithms 1110 that are either in the Robot's 1002 microprocessor or from the A-Medicate server-based Machine Learning algorithms 1018 accessing the Federated database and the A-Medicare Cloud Infrastructure 1020. All other commands 1108 and corresponding task executions 1114 are learned without further programming.

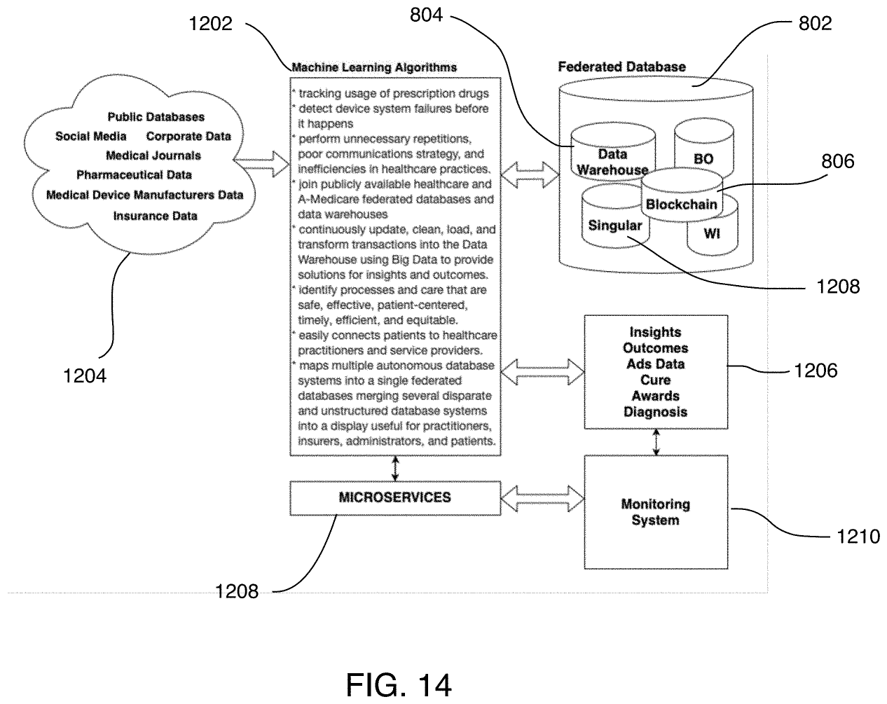

In some implementations, regarding the Process and Machine Learning Program Warehouse, the system creates and utilizes thousands of revolutionary and novel machine learning algorithms 1202, as may be appreciated in FIG. 14. These algorithms are ran against third-party data 1204 that can track misuse, underuse, or overuse prescription drugs, detect device system failures before it happens, perform unnecessary repetitions, poor communications strategy, and inefficiencies in healthcare practices. The algorithms 1202 merge publicly available and third-party healthcare data 1204 and A-Medicare federated databases 802, blockchain 806, and warehouses 804 to continuously update, clean, load, and transform transactions into the Data Warehouse 804 using third-party data 1204 to provide solutions for insights and outcomes 1206. They identify processes and care that are safe, effective, patient-centered, timely, efficient, and equitable. They also easily connect patients to healthcare practitioners and service providers.

The algorithms also map multiple autonomous database systems into a single federated databases 1208 merging several disparate and unstructured database systems into a display useful for practitioners, insurers, administrators, and patients. This allows the system to take in raw data from many servers in different formats, convert it to a singular format that can then be applied across the system. The Machine Learning Algorithms 1202 also integrate bi-directionally with microservice 1208 and are influenced by the monitoring system 1210.

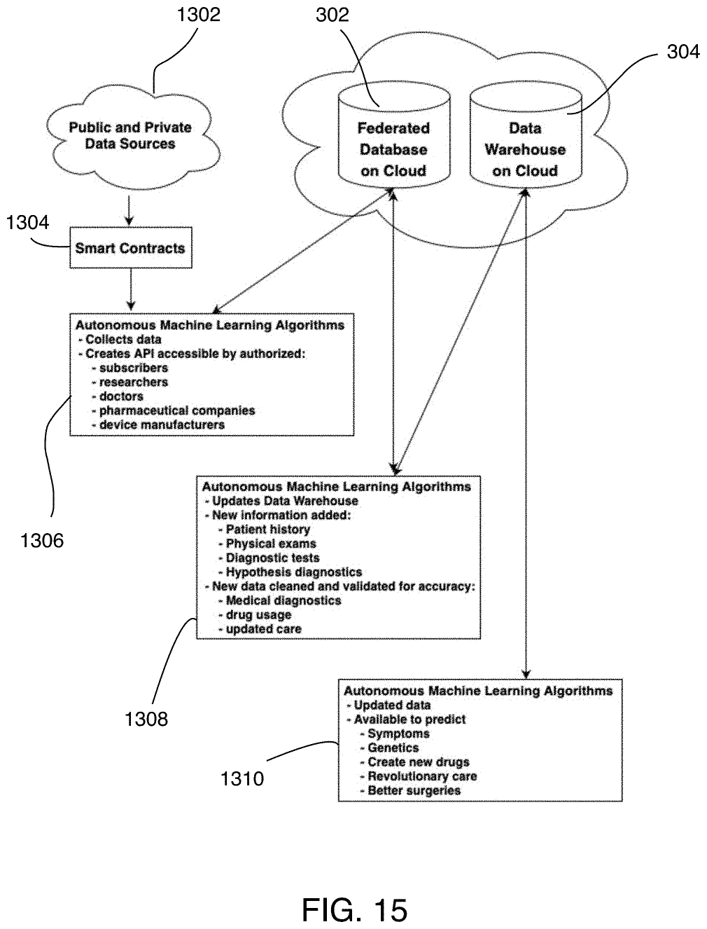

FIG. 15 illustrates the integration of autonomous machine learning algorithms with various functionality of the system. Data from various A-Medicare processes and other third-party sources 1302 public and private (with prior smart contracts 1304) are collected into a secure Data Warehouse 304 and federated databases 302. Thousands of machine learning algorithms 1306/1308/1310 are created by A-Medicare Data and Machine Learning Scientists and published in a user-friendly interface accessible by authorized subscribers, researchers, doctors, and other participants. Autonomous Machine Learning Algorithms 1306 collect this data, and through an API the data is further accessible to pharmaceutical companies, and device manufacturers, in addition to the subscribers, researchers, and doctors.

This Data Warehouse 304 using third-party technology is continually updated and new information from the autonomous machine learning algorithms 1308 such as patient history, physical exams, diagnostic test, and hypothesis diagnosis is added. These new data are all validated and cleaned by further collections using Machine Learning and best practices making medical diagnostics, drug usage, and other care more accurate. These data are accessible by using thousands of readily available machine learning algorithms 1310 to predict symptoms, genetics of the participants, and how it will respond to new drugs and revolutionary care or surgeries.

An exemplary aspect of the system is that the use of data may influence the suggested treatment of a patient, which may include variables like availability of drugs, matching organs, locations of available medical supplies, organs, and medical capabilities in some regions of the country. This allows the machine learning algorithms to provide real time suggestions on actually available treatments and therapeutics that can be administered to a specific patient in a specific region at any given time.

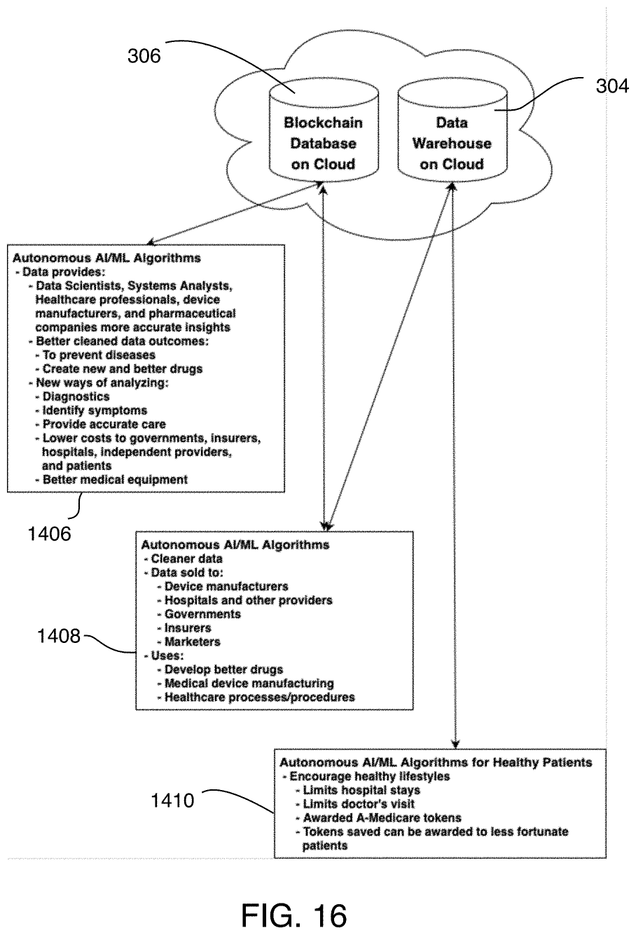

FIG. 16 illustrates an exemplary embodiment the integration of autonomous machine learning algorithms with various functionality of the system. Use of third-party, Federated, Data Warehouse 304, Blockchain databases 306, and artificial intelligence, coupled with the latest machine learning algorithms 1406, can provide Data Scientists, Systems Analysts, Healthcare professionals, device manufacturers, and pharmaceuticals a more accurate insights and outcomes. Using newer digital transformation tools, the results and outcomes can prevent diseases, create new and better drugs, provide new ways of analyzing diagnostic test, identifying symptoms accurately, and provide the accurate care and its costs, and influence the invention of new medical equipment. Other algorithms 1408 provide cleaner data, which is easier to implement in the system's uniform protocol. The data output from the algorithm 1408 can be sold to device manufacturers, hospitals and providers, governments, insurance entities, and marketers for use with developing better drugs, better medical device manufacturing, and better healthcare processes and procedures. Further, some algorithms 1410 encourage healthier lifestyles to limit hotel stays, limit doctor visits, award incentives, such as tokens, and encourage use of excess tokens to increase the health of less fortunate users.

This amalgamation of newer digital transformation tools, in the long run, saves cost to the government, insurers, hospitals, independent providers, and patients. These set of newer digital transformation tools accessing the Federated database provide all the necessary information that follows PHI, PII, HIPAA, Cybersecurity policy, and other government policies. The digital transformation tools can be used by Information Technology and Software Engineers to develop blockchain-based, better secure code applications and software modules to manage doctor's appointments, insurance, provider, or host services such as surgeries, doctor's visits, ICU, ER, medications, specialists, and drugs. Further, these tools provide a comparison of quality care, various services, insurance premiums, cost of hospitalizations, and the right drugs for identified symptoms.

Machine learning algorithms 1408 can provide insights and outcomes of user behaviors in accessing A-Medicare platform's public as well as the user's private information. Captured information can be sold to pharmaceutical companies, device manufacturers, hospitals, governments, insurance, marketers, and other providers. This can be used, as an example, to develop better drugs, medical devices, and healthcare processes.

Special programs, policies, and smart contracts 1304 can be developed to award A-Medicare subscribers who practice healthy lifestyles using algorithms 1410. Cryptocurrency tokens can be awarded to this group of patients or subscribers who have limited use of hospital stays and doctor's visits. The same group of users can share, if desired, their tokens to patients who badly need financial support for their surgeries, operations, and other medical needs such as home health.

The system is also designed to increase competition amongst healthcare actors. Healthy competition is good for the healthcare industry and patient-centered support by providers. Competition leads further to cooperation, partnership, and joint research, as well as the development of better drugs, medical devices, accurate care, and other technologies. Competing products bring down costs of drugs, medical services, and other healthcare costs without compromising quality care.

By prior agreement and/or use of smart contracts 1304 with competing entities, the system publishes information shared to competing healthcare actors can speed up care, accurate use of drugs, validation of peer-to-peer medical journal, and identifies and shares potential diseases such as novel viruses and the best tools for contact tracing.

A-Medicare as a platform for healthcare will provide leadership by establishing alliance and convening with insurance companies, providers, pharmaceuticals, device manufacturers, financial institutions, insurers, governments, and aid organizations to establish policies and standards in the ever-growing complexities in the healthcare practice worldwide.

A-Medicare's server-based processes and databases is hosted on a cloud Infrastructure as a Service (IaaS). Singular databases and Data Warehouses are PostgreSQL-based services and blockchain based distributed ledger technology (DLT). These databases securely store HIPAA mandated PHI and PII data, patient, provider, diagnosis, research, financial, advertisement, insurance, and related healthcare data. Thousands of software web services and other processes based on SQL and machine learning algorithms drive A-Medicare platform hosting healthcare data managed by PostgreSQL.

Some implementations of the system include protocols for reuse personal medical devices, surplus tokens, and unused drugs. Personal medical devices such as walkers, oxygen tanks, that are in perfect condition are encouraged for reuse to patients in need. Reuse of this devices will eliminate unnecessary trash as well as help for the environment and climate change. By sharing these devices givers will be awarded tokens for use in future doctor's visit and hospital care as examples. These tokens are pooled in a user's account with other tokens received from other activities, such as engaging in ad-based content.

Patients, subscribers, and other online users who have surplus A-Medicare tokens are encouraged to voluntarily awards tokens to be used by patients who have less fortunate financial capability or insufficient tokens for necessary procedures and other healthcare needs. Unused and unexpired prescription drugs can be authorized by A-Medicare doctors for use by patients especially in times of inventory stock crisis during periods of pandemic. Givers of tokens, unused drugs and medical devices are awarded additional tokens as a good Samaritan and as a token of appreciation by the patients and A-Medicare.

In some implements of the invention, sharing of medical records helps keep the system up to date and both users and caregivers informed. Medical records, diagnosis, drug usage, and the type of care patients received can be shared to advertisers, pharmaceutical, drug and medical device manufacturers by scrubbing PHI and PII data. It is hoped that sharing these types of data will lead to faster cure, discovery of new drugs, and improved healthcare processes and procedures. Extreme care will be religiously followed to ensure that no PHI, PII, and diagnostics data slipped to unauthorized personnel.

Sharing of healthcare data will also be used to develop a matching algorithmic engine to match a patient with possible organ donors sourced worldwide. When the time comes, search A-Medicare federated database for the nearest organ donor's hospital or medical clinic who has the skill and experience to transfer the organ to the legal recipient considering the legal, ethical, and political sensitivities.

Some implements of the invention provides for transparency in health insurance claims. The system expedites transparency in every single health insurance claim made by a citizen into a doctor's office, specialist, hospital or medical facility (private or public) nationwide. In some embodiments, this may include publishing of pricing by healthcare providers and pharmaceuticals, but may also include storing of health receipts from user's transactions wherein the itemized expenses are processed in order to compile averages that may be broken down by nation, state, region, or hospital/caregiver.

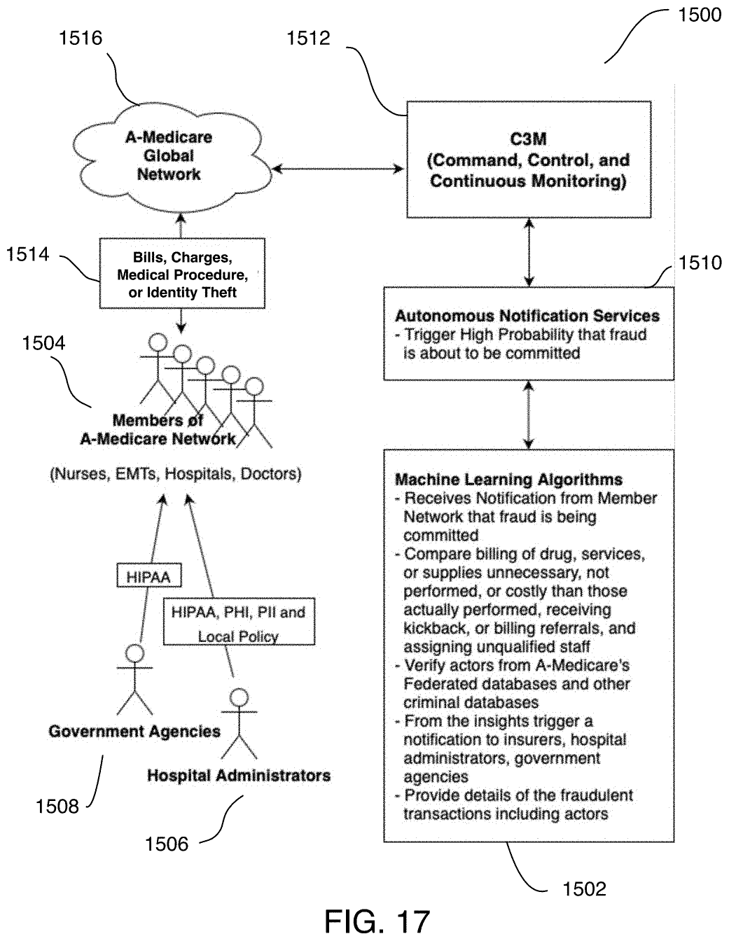

FIG. 17 illustrates a fraud early warning system. With the information gathered from the system for transparency in health insurance claims, a Fraud Early Warning System 1500 may be implemented using machine learning algorithms 1502 to counteract improper practices and procedures thereby preventing fraud by medical exams, unnecessary tests, over-prescription medication, and over-billing. The A-Medicare Fraud Early Warning System creates a network of providers 1504, hospital administrators 1506, doctors, insurers, government agencies 1508, and other actors. The system uses A-Medicare Federated database and other criminal databases to compare with fraudulent transactions about to be or has been committed. Autonomous Machine Learning algorithms 1502 detect, trigger, and notify all concerned actors through an autonomous notification system 1510. The Autonomous notifies based on triggers of high-probability situations where fraud is about to be committed. The autonomous notification system 1510 communicates bi-directionally with the and C3M 1512.

The system also compares the patient's symptoms with the patient's treatment and billing, charges, medical procedures, or any identity theft 1514 reported to the A-Medicare global network 1516 and the machine learning algorithms 1502 identify billing of unnecessary drugs, services not performed, supplies unnecessary, or costly that those actually performed, receiving kickbacks, billing referrals, and assigning unqualified staff. This information is compared to allowable price range from the A-Medicare Federated database and other established billing rates from corporate billers. The information is then used to manually verify and analyze the fraudulent transaction for reporting. The system documents details of the fraudulent transactions and the bad actors. From the insights and outcomes, the system will trigger a notification from the notification system 1510 to insurers, hospital administrators, and government agencies.

Those skilled in the art will readily recognize, in light of and in accordance with the teachings of the present invention, that any of the foregoing steps and/or system modules may be suitably replaced, reordered, removed and additional steps and/or system modules may be inserted depending upon the needs of the particular application, and that the systems of the foregoing embodiments may be implemented using any of a wide variety of suitable processes and system modules, and is not limited to any particular computer hardware, software, middleware, firmware, microcode and the like.

For any method steps described in the present application that can be carried out on a computing machine, a typical computer system can, when appropriately configured or designed, serve as a computer system in which those aspects of the invention may be embodied. Such computers referenced and/or described in this disclosure may be any kind of computer, either general purpose, or some specific purpose computer such as, but not limited to, a workstation, a mainframe, GPU, ASIC, etc. The programs may be written in C, or Java, Brew or any other suitable programming language. The programs may be resident on a storage medium, e.g., magnetic or optical, e.g., without limitation, the computer hard drive, a removable disk or media such as, without limitation, a memory stick or SD media, or other removable medium. The programs may also be run over a network, for example, with a server or other machine sending signals to the local machine, which allows the local machine to carry out the operations described herein.

System Integration with Client/Server Systems

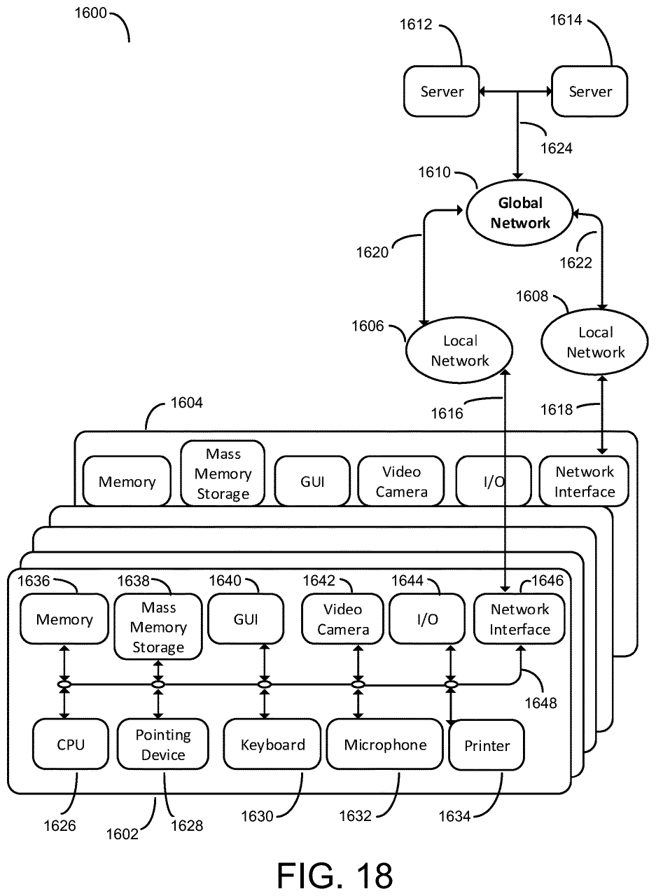

FIG. 18 is a block diagram depicting an exemplary client/server system which may be used by an exemplary web-enabled/networked embodiment of the present invention.

A communication system 1600 includes a multiplicity of clients with a sampling of clients denoted as a client 1602 and a client 1604, a multiplicity of local networks with a sampling of networks denoted as a local network 1606 and a local network 1608, a global network 1610 and a multiplicity of servers with a sampling of servers denoted as a server 1612 and a server 1614.

Client 1602 may communicate bi-directionally with local network 1606 via a communication channel 1616. Client 1604 may communicate bi-directionally with local network 1608 via a communication channel 1618. Local network 1606 may communicate bi-directionally with global network 1610 via a communication channel 1620. Local network 1608 may communicate bi-directionally with global network 1610 via a communication channel 1622. Global network 1610 may communicate bi-directionally with server 1612 and server 1614 via a communication channel 1624. Server 1612 and server 1614 may communicate bi-directionally with each other via communication channel 1624. Furthermore, clients 1602, 1604, local networks 1606, 1608, global network 1610 and servers 1612, 1614 may each communicate bi-directionally with each other.

In one embodiment, global network 1610 may operate as the Internet. It will be understood by those skilled in the art that communication system 1600 may take many different forms. Non-limiting examples of forms for communication system 1600 include local area networks (LANs), wide area networks (WANs), wired telephone networks, wireless networks, or any other network supporting data communication between respective entities.

Clients 1602 and 1604 may take many different forms. Non-limiting examples of clients 1602 and 1604 include personal computers, personal digital assistants (PDAs), cellular phones and smartphones.

Client 1602 includes a CPU 1626, a pointing device 1628, a keyboard 1630, a microphone 1632, a printer 1634, a memory 1636, a mass memory storage 1638, a GUI 1640, a video camera 1642, an input/output interface 1644 and a network interface 1646.

CPU 1626, pointing device 1628, keyboard 1630, microphone 1632, printer 1634, memory 1636, mass memory storage 1638, GUI 1640, video camera 1642, input/output interface 1644 and network interface 1646 may communicate in a unidirectional manner or a bi-directional manner with each other via a communication channel 1648. Communication channel 1648 may be configured as a single communication channel or a multiplicity of communication channels.

CPU 1626 may be comprised of a single processor or multiple processors. CPU 1626 may be of various types including micro-controllers (e.g., with embedded RAM/ROM) and microprocessors such as programmable devices (e.g., RISC or SISC based, or CPLDs and FPGAs) and devices not capable of being programmed such as gate array ASICs (Application Specific Integrated Circuits) or general-purpose microprocessors.

As is well known in the art, memory 1636 is used typically to transfer data and instructions to CPU 1626 in a bi-directional manner. Memory 1636, as discussed previously, may include any suitable computer-readable media, intended for data storage, such as those described above excluding any wired or wireless transmissions unless specifically noted. Mass memory storage 1638 may also be coupled bi-directionally to CPU 1626 and provides additional data storage capacity and may include any of the computer-readable media described above. Mass memory storage 1638 may be used to store programs, data and the like and is typically a secondary storage medium such as a hard disk. It will be appreciated that the information retained within mass memory storage 1638, may, in appropriate cases, be incorporated in standard fashion as part of memory 1636 as virtual memory.

CPU 1626 may be coupled to GUI 1640. GUI 1640 enables a user to view the operation of computer operating system and software. CPU 1626 may be coupled to pointing device 1628. Non-limiting examples of pointing device 1628 include computer mouse, trackball and touchpad. Pointing device 1628 enables a user with the capability to maneuver a computer cursor about the viewing area of GUI 1640 and select areas or features in the viewing area of GUI 1640. CPU 1626 may be coupled to keyboard 1630. Keyboard 1630 enables a user with the capability to input alphanumeric textual information to CPU 1626. CPU 1626 may be coupled to microphone 1632. Microphone 1632 enables audio produced by a user to be recorded, processed and communicated by CPU 1626. CPU 1626 may be connected to printer 1634. Printer 1634 enables a user with the capability to print information to a sheet of paper. CPU 1626 may be connected to video camera 1642. Video camera 1642 enables video produced or captured by user to be recorded, processed and communicated by CPU 1626.

CPU 1626 may also be coupled to input/output interface 1644 that connects to one or more input/output devices such as such as CD-ROM, video monitors, track balls, mice, keyboards, microphones, touch-sensitive displays, transducer card readers, magnetic or paper tape readers, tablets, styluses, voice or handwriting recognizers, or other well-known input devices such as, of course, other computers.

Finally, CPU 1626 optionally may be coupled to network interface 1646 which enables communication with an external device such as a database or a computer or telecommunications or internet network using an external connection shown generally as communication channel 1616, which may be implemented as a hardwired or wireless communications link using suitable conventional technologies. With such a connection, CPU 1626 might receive information from the network, or might output information to a network in the course of performing the method steps described in the teachings of the present invention.

System Integration with Web-Enablement and/or Networking

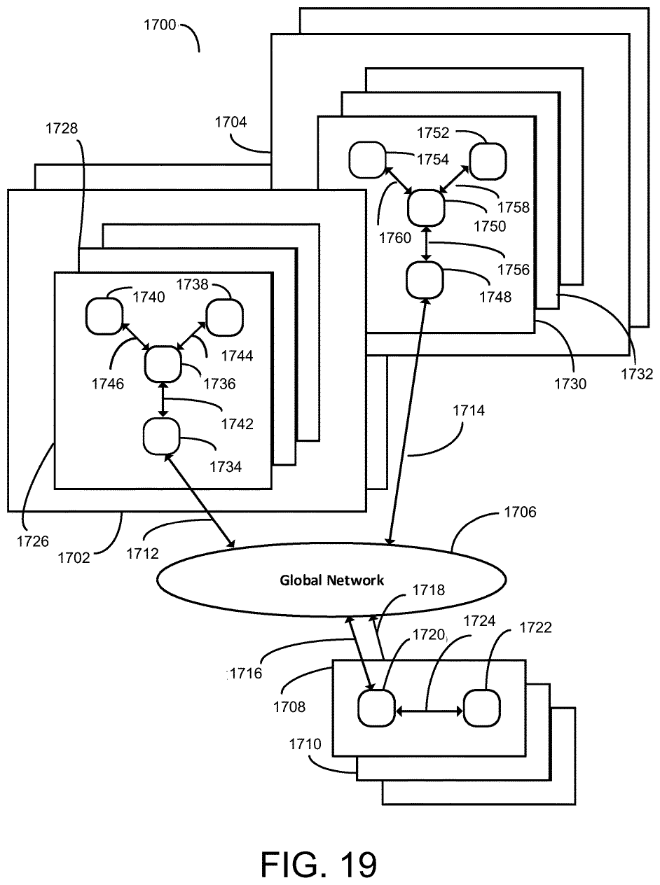

FIG. 19 illustrates a block diagram depicting a conventional client/server communication system, which may be used by an exemplary web-enabled/networked embodiment of the present invention.

A communication system 1700 includes a multiplicity of networked regions with a sampling of regions denoted as a network region 1702 and a network region 1704, a global network 1706 and a multiplicity of servers with a sampling of servers denoted as a server device 1708 and a server device 1710.

Network region 1702 and network region 1704 may operate to represent a network contained within a geographical area or region. Non-limiting examples of representations for the geographical areas for the networked regions may include postal zip codes, telephone area codes, states, counties, cities and countries. Elements within network region 1702 and 1704 may operate to communicate with external elements within other networked regions or within elements contained within the same network region.

In some implementations, global network 1706 may operate as the Internet. It will be understood by those skilled in the art that communication system 1700 may take many different forms. Non-limiting examples of forms for communication system 1700 include local area networks (LANs), wide area networks (WANs), wired telephone networks, cellular telephone networks or any other network supporting data communication between respective entities via hardwired or wireless communication networks. Global network 1706 may operate to transfer information between the various networked elements.

Server device 1708 and server device 1710 may operate to execute software instructions, store information, support database operations and communicate with other networked elements. Non-limiting examples of software and scripting languages which may be executed on server device 1708 and server device 1710 include C, C++, C# and Java.

Network region 1702 may operate to communicate bi-directionally with global network 1706 via a communication channel 1712. Network region 1704 may operate to communicate bi-directionally with global network 1706 via a communication channel 1714. Server device 1708 may operate to communicate bi-directionally with global network 1706 via a communication channel 1716. Server device 1710 may operate to communicate bi-directionally with global network 1706 via a communication channel 1718. Network region 1702 and 1704, global network 1706 and server devices 1708 and 1710 may operate to communicate with each other and with every other networked device located within communication system 1700.

Server device 1708 includes a networking device 1720 and a server 1722. Networking device 1720 may operate to communicate bi-directionally with global network 1706 via communication channel 1716 and with server 1722 via a communication channel 1724. Server 1722 may operate to execute software instructions and store information.

Network region 1702 includes a multiplicity of clients with a sampling denoted as a client 1726 and a client 1728. Client 1726 includes a networking device 1734, a processor 1736, a GUI 1738 and an interface device 1740. Non-limiting examples of devices for GUI 1738 include monitors, televisions, cellular telephones, smartphones and PDAs (Personal Digital Assistants). Non-limiting examples of interface device 1740 include pointing device, mouse, trackball, scanner and printer. Networking device 1734 may communicate bi-directionally with global network 1706 via communication channel 1712 and with processor 1736 via a communication channel 1742. GUI 1738 may receive information from processor 1736 via a communication channel 1744 for presentation to a user for viewing. Interface device 1740 may operate to send control information to processor 1736 and to receive information from processor 1736 via a communication channel 1746. Network region 1704 includes a multiplicity of clients with a sampling denoted as a client 1730 and a client 1732. Client 1730 includes a networking device 1748, a processor 1750, a GUI 1752 and an interface device 1754. Non-limiting examples of devices for GUI 1738 include monitors, televisions, cellular telephones, smartphones and PDAs (Personal Digital Assistants). Non-limiting examples of interface device 1740 include pointing devices, mousse, trackballs, scanners and printers. Networking device 1748 may communicate bi-directionally with global network 1706 via communication channel 1714 and with processor 1750 via a communication channel 1756. GUI 1752 may receive information from processor 1750 via a communication channel 1758 for presentation to a user for viewing. Interface device 1754 may operate to send control information to processor 1750 and to receive information from processor 1750 via a communication channel 1760.

For example, consider the case where a user interfacing with client 1726 may want to execute a networked application. A user may enter the IP (Internet Protocol) address for the networked application using interface device 1740. The IP address information may be communicated to processor 1736 via communication channel 1746. Processor 1736 may then communicate the IP address information to networking device 1734 via communication channel 1742. Networking device 1734 may then communicate the IP address information to global network 1706 via communication channel 1712. Global network 1706 may then communicate the IP address information to networking device 1720 of server device 1708 via communication channel 1716. Networking device 1720 may then communicate the IP address information to server 1722 via communication channel 1724. Server 1722 may receive the IP address information and after processing the IP address information may communicate return information to networking device 1720 via communication channel 1724. Networking device 1720 may communicate the return information to global network 1706 via communication channel 1716. Global network 1706 may communicate the return information to networking device 1734 via communication channel 1712. Networking device 1734 may communicate the return information to processor 1736 via communication channel 1742. Processor 1736 may communicate the return information to GUI 1738 via communication channel 1744. User may then view the return information on GUI 1738.

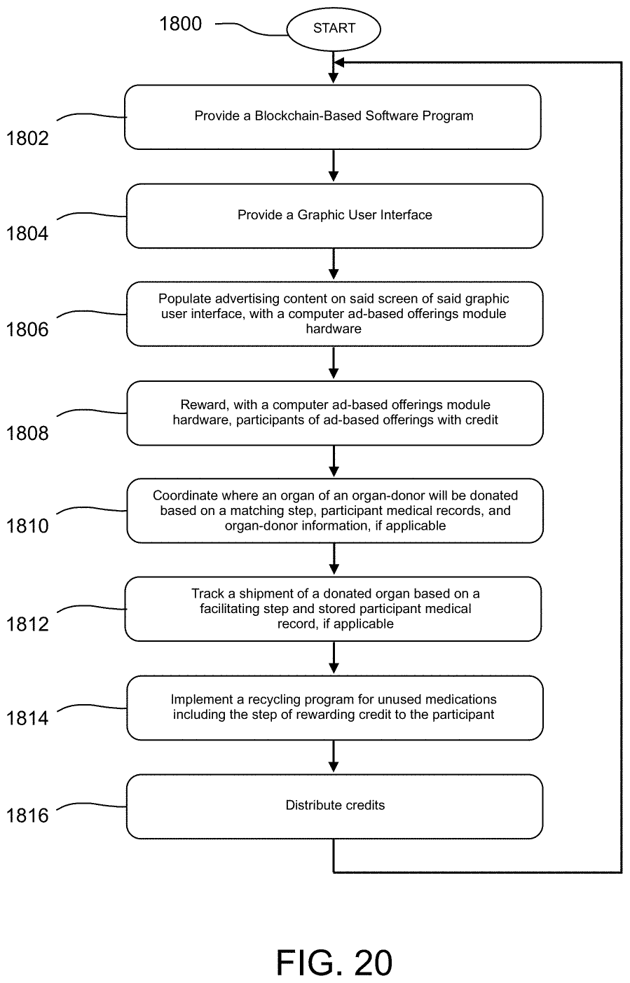

Having described the infrastructure and components above, an exemplary embodiment of the present invention provides a for method of a unified platform for universal healthcare and related services. FIG. 20 illustrates a flowchart of an exemplary method of a unified platform for universal healthcare and related services, in accordance with an embodiment of the present invention. Referring to the present embodiment, a method begins at a start operation 1800. Providing a blockchain-based software program, stored in a non-transitory computer-readable storage digital medium and being executed by one or more processors of a computer-based system is accomplished in operation 1802. Upon providing a blockchain-based software program in operation 1802, the method continues by providing a graphic user interface coupled to the non-transitory computer-readable storage digital medium and the processor in operation 1804. Populating advertising content on the screen of the graphic user interface, with a computer ad-based offerings module hardware, at least partially based on successful consumption of the advertising content as indicated by a completion of a survey by the participant and return of same to a private-party advertiser, wherein the participant earns credit in return from consumption of the advertising content is included in operation 1806. Rewarding, with the computer ad-based offerings module hardware, participants with credit towards the participant's healthcare bills or premiums is accomplished in operation 1808. Coordinating where an organ of the organ-donor will be donated based on the matching step, the participant medical records, and organ-donor information is accomplished in operation 1810. Tracking a shipment of the donated organ based on the facilitating step and the stored participant medical record is accomplished in operation 1812. The tracking step 1812 comprises tracking an organ pick-up location and a delivery of the organ to a destination location. Creating a recycling program for unused medications including the step of rewarding credit to the participant, thereby motivating participants to recycle unused medications to local pharmacies is accomplished in operation 1814. Distributing credits is accomplished in operation 1816, wherein the distributed credits include at least one of the credits obtained for participating in viewing advertising content and the credits obtained from participating in the recycling program, in which at least a portion of the credit is distributed to multiple entities including the user, the medical insurer, the pharmacy, and governmental health agencies, wherein the user's portion of credit is recorded in a virtual bank of credits to be selectably used on medical bills and services.

In some embodiments, the blockchain-based program 1802 includes an open, distributed ledger that is configured to record a transaction between the organ-donor and participant including the organ-donor and participant in a verifiable and permanent way.

In some embodiments, the blockchain-based program 1802 illustrated in FIG. 20 is further configured to perform the steps of processing and prioritizing a health insurance claim in at least one of a public or private doctor's office, a public or private specialist, and a public or private hospital or medical facility nationwide.

In some embodiments, the advertising content 1806 illustrated in FIG. 20 further includes a digital platform for advertisements where the private party advertiser pays to advertise their products to specific target audiences with the survey at an end of each advertisement.

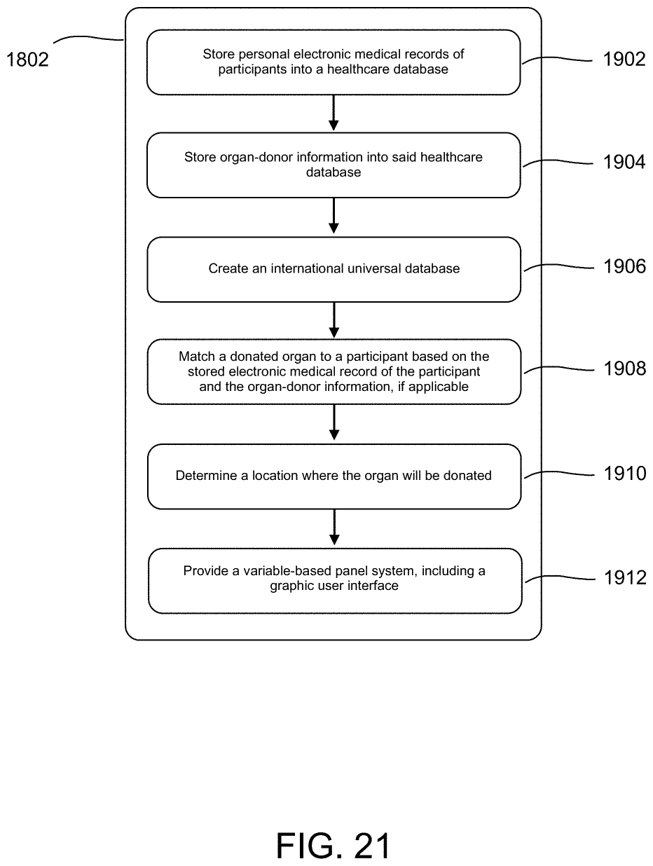

FIG. 21 illustrates a flow chart of a subroutine of the blockchain-based software program of operation 1802 shown in FIG. 20, and is configured to perform the operation 1902 of storing personal electronic medical records of participants into a healthcare database as a growing list of the personal electronic medical records including personal electronic medical record blocks that are linked together using cryptography, wherein each personal electronic medical record block contains a cryptographic hash of the previous personal electronic medical record block. Once the personal electronic medical is recorded, the personal electronic medical record in any given block cannot be altered retroactively without alteration of all subsequent personal electronic medical record blocks, which requires consensus of a network majority. Following operation 1902, the operation 1904 of storing an organ-donor information into the healthcare database as a growing list of the organ-donor information including organ-donor information blocks that are linked together using cryptography, wherein each organ-donor information block contains a cryptographic hash of the previous organ-donor information block is accomplished. Once the organ-donor information is recorded, the organ-donor information in any given block cannot be altered retroactively without alteration of all subsequent organ-donor information blocks, which requires consensus of a network majority. Creating an international universal database, wherein the international universal database is configured to connect the organ-donor with a recipient at least nationally or worldwide is accomplished in operation 1906. Matching a donated organ to a participant based on the stored electronic medical record of the participant and the organ-donor information is accomplished in operation 1908. Determining a location where the organ will be donated based on the matching step is accomplished in operation 1910. Providing a variable-based panel system, including a graphic user interface, where a user can manage at least one of, a doctor's appointment, the electronic medical record, healthcare insurance, hospital visits, medications, and pharmacy, wherein the variable-based panel is configured to be locked with an assigned ID and Social Security Number is accomplished in operation 1912.

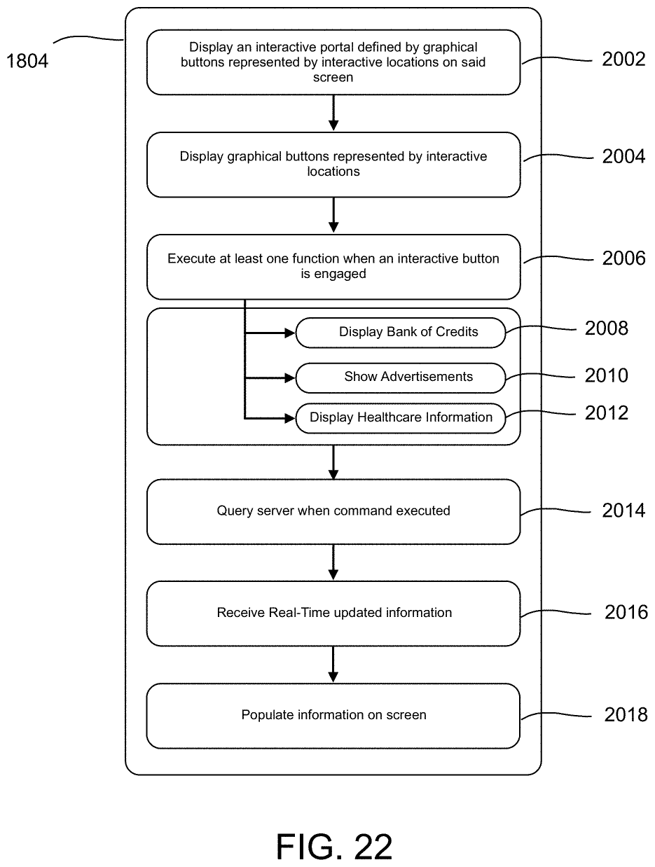

FIG. 22 illustrates a flow chart of the subroutine of operation 1804 in FIG. 20. The graphic user interface displays a graphical output of the computer-based-system on a screen which includes displaying an interactive portal defined by graphical buttons represented by interactive locations on the screen as shown in operation 2002, and displaying the graphical buttons represented by the interactive locations as shown in operation 2004, wherein, when engaged, the interactive buttons executes at least one function 2006, including providing a menu of executable sub functions 2020, including displaying a bank of credits 2008, showing advertisements 2010, displaying healthcare information 2012, whereby selection of each option executes commands to query a server 2014, receive real-time information 2016, and populate the information on the screen 2018.

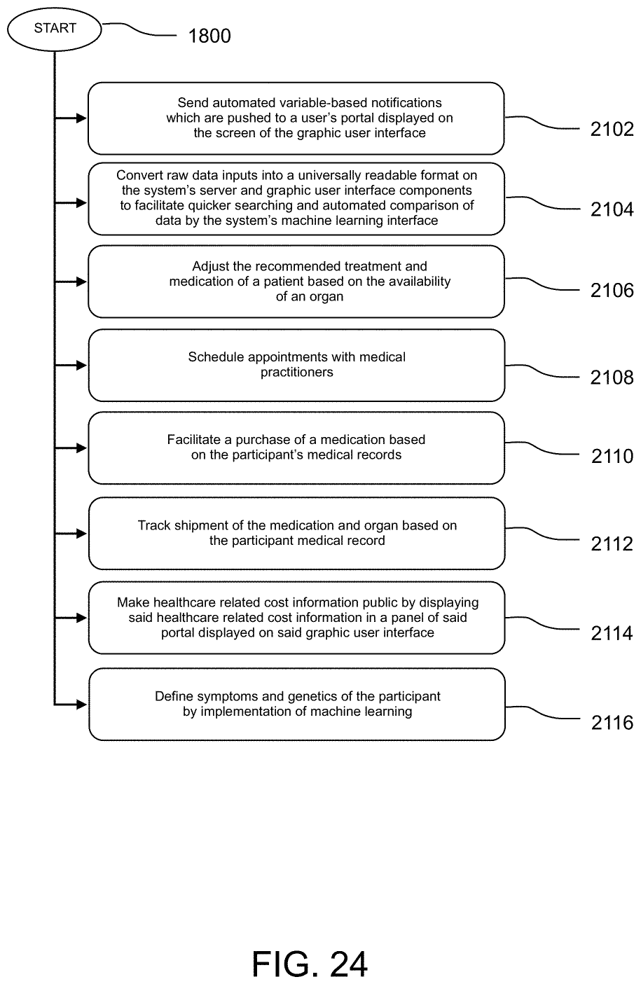

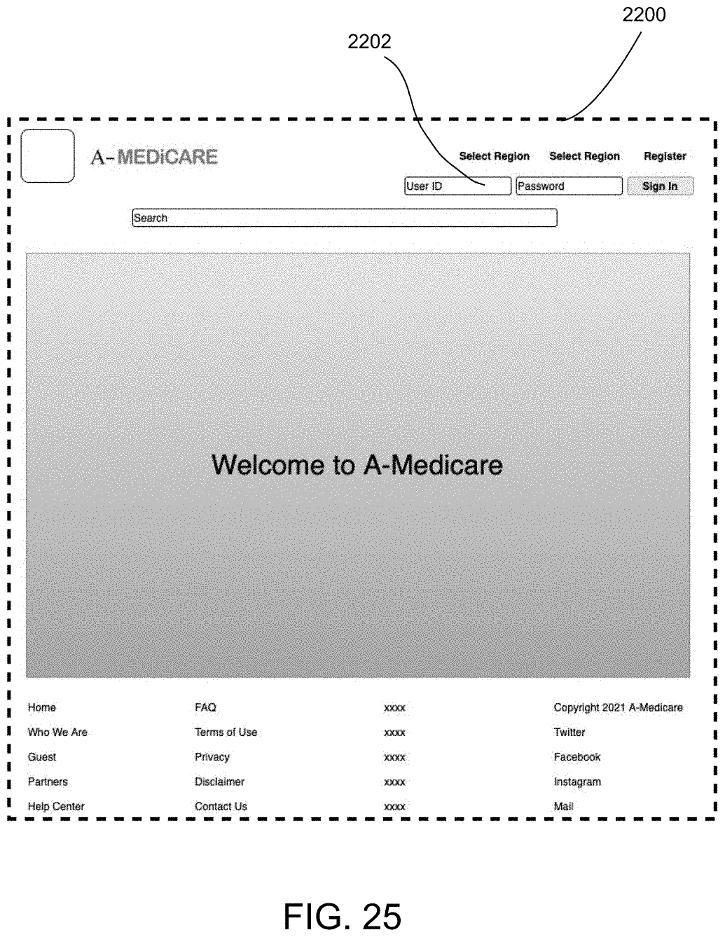

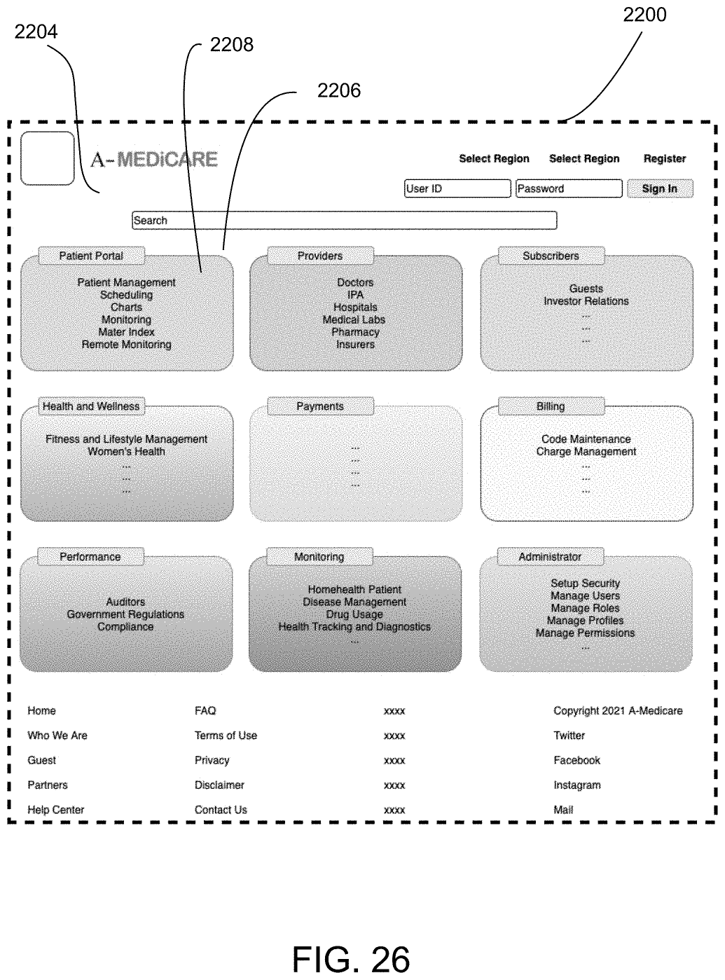

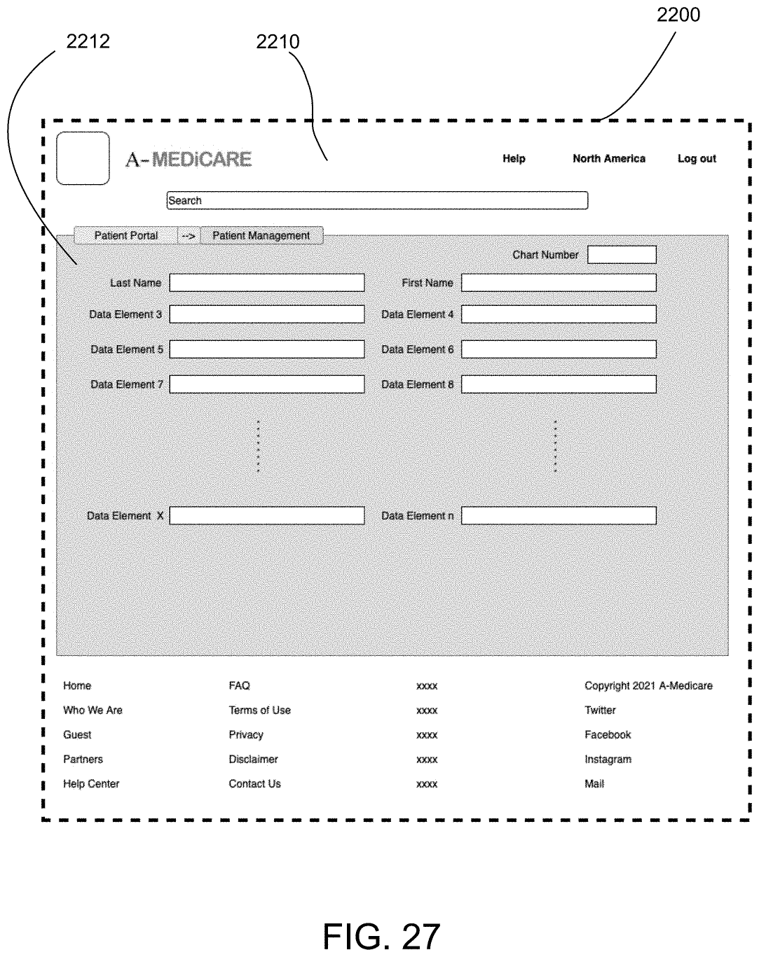

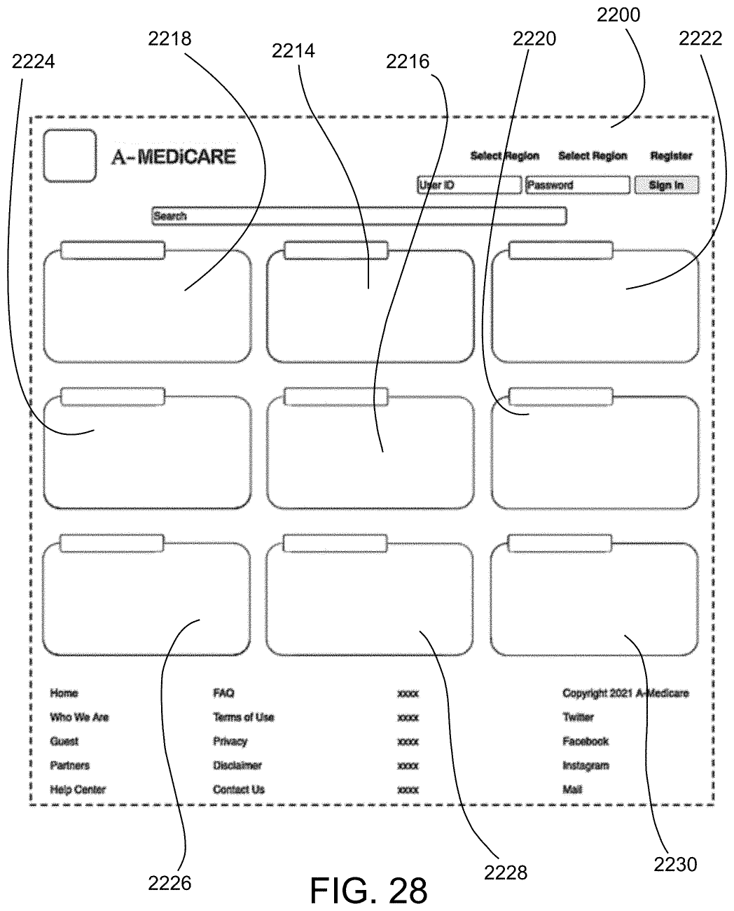

FIGS. 25, 26, and 27 illustrate the visual correlative to some exemplary operations discussed in the flow chart of FIG. 24. In the exemplary embodiment shown in FIG. 25, a user is provided an interactive portal login module 2202 on the graphic user interface 2200. Upon authenticating the user and logging the user in, a graphic user interface 2200 will display the user portal 2204 having several buttons 2208 representing interactive locations 2206, as shown in FIG. 26, that, once clicked, will execute a sub routine, such as taking the user to a specific subpage. FIG. 27 illustrates the Patent Management section of the Patient Portal subpage 2210 on the graphic user interface 2200, which allows a user to input new information. Other subpages may be executed from other buttons in the user portal 2204. This user portal allows a user a unified area to monitor and view all related information regarding the user's or user's patient's healthcare information. This may also include ad-based credit offerings, billing functions, and monitoring, among others, which are all integrated in one place, though the information may be taken from multiple sources, these areas are further discussed in Reference to FIG. 28. Information taken from other sources may be converted to an integratable universal template 2212, such as that shown in FIG. 27. While FIG. 27 includes a fillable template from a user, it should also be appreciated that the system also receives information from non-interactive templates, which are then processed by the system and the information therein is read and uploaded to a patient's portal without direct action from the user.

In furtherance and in combination with the above-described system, the GUI combines features that allow for a universal portal access for modules and integrated programs, including tracking information relating to medication and organ donation, health information, a universal template for users to input data and display said data in a universal format, and for a user to see things including ads and credit balance from reviewing ads.

FIG. 28 illustrates an exemplary embodiment of the graphic user interface 2220, wherein a plurality of modules are present, each representing a sub functional program and interactive location, such as a clickable button, displaying each area. For example, these modules may include an ad module 2214, organ tracking module 2216, recommended health plan module 2218, credit balance module 2220, medical records module 2222, integration module 2224 for interaction with a user's personal or voice assistant, imaging uploading and processing module 2226, medical billing module 2228, and healthcare provider information module 2230. It should be appreciated that each of these modules display data received from multiple sources in real-time for user interaction and are interactive that, when clicked, will take a user to a sub screen of the GUI 2220, such as that shown in FIG. 27.

This invention is a practical application of the principles of software integration because it combines the different features into a single portal, wherein they are able to interact and influence one another. For example, a plan of treatment may update automatically if an organ donation is not possible before a predesignated threshold. Further, the exemplary embodiment provides a technical solution to a technical problem, wherein the problem stems from a dissociated and/or fragmented offering of healthcare “related” applications. While there are healthcare systems in place for medical needs, this invention allows for a patient-centered solution that includes things beyond the scope of healthcare specific information, such as the ad-funded credits, which allow a user to integrate the funds into use on medical bills and the like, but are not in and of themselves (referring to the credits) healthcare products. Further, it should be appreciated from the solutions mentioned above that the solutions cannot be completed in the mind of a user, as they require variable input from multiple sources that interact with variable real-time data from other sources for a real-time output that is affected simultaneously from the numerous data inputs.

This system is available to users of all levels, wherein each user may receive a differently composed graphic user interface 2200. For example, a patient may receive a layout as shown and described in the above FIG. 28. However, a healthcare professional may receive a portal with areas to upload notes, scan notes into digital text format, upload images, run a machine-learning function to analyze images and notes to reduce time spent on diagnosis or provided an immediate second opinion, manually upload organ information donation availability and/or tracking information if not controlled by the AI management system, send secure messages to patients, and/or upload and/or modify billing. Likewise, advertisers may receive a simpler portal, wherein the only feature may be to upload advertising media and filter target characteristics of users for receipt of said media.

Because a plurality of individuals that may affect a single user's portal, the system accepts inputs and transforms them into universal data format, including universally implemented metadata read by the end-user portal program, and will provide real-time information that quickly adapts to new information received, thereby modifying displayed information, including recommendations, credit balances, and healthcare information. Further, because the system includes an AI assisted machine learning algorithm, the system is monitored and updated continuously, providing information beyond user input, wherein such additional information is neither uploaded or recommended by a physical user.

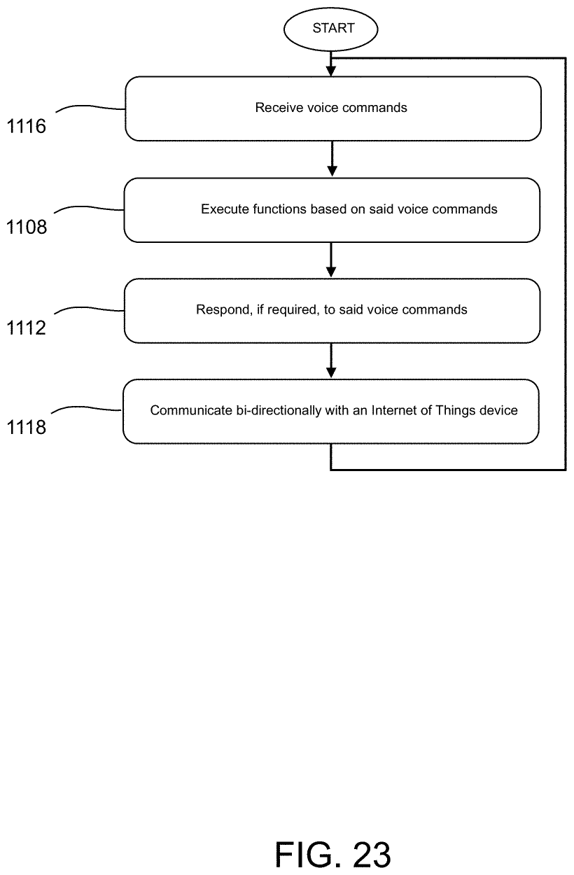

In some embodiments, the blockchain-based program 1802 is further configured to perform the following steps of providing a virtual personal health assistant (“VPHA”), wherein the virtual personal health assistant is configured to be operable for following voice and/or text commands 1108 to provide information and assistance, as may be appreciated in the flow chart illustrated in FIG. 23.

In some embodiments, further illustrated in FIG. 23, the virtual personal health assistant is a home-based robot 1002 connected to the blockchain-based program 1802 through a home portal 1104, and the robot 1002 including a memory on which the robot 1002 includes instructions thereon for receiving voice commands 1116 and execute functions based on the commands 1108, and communicating bi-directionally 1118 with an Internet of Things device 220 including at least one of a smart wearable and chip imbedded into a user's skin.

FIG. 24 shows additional steps to the exemplary method of a unified platform for universal healthcare and related services, in accordance with the embodiment illustrated in FIG. 20. In some embodiments, the exemplary method of a unified platform for universal healthcare and related services, in accordance with an embodiment of the present invention illustrated in FIG. 20 further includes the operation 2102 of sending automated variable-based notifications which are pushed to a user's portal displayed on the screen of the graphic user interface, wherein notifications result from at least real-time changes to a patient's information including conditions reported by a review from machine learning, conditions observed by wearable and Internet of Things technology, availability of organs, and changes in diagnosis.

In some embodiments, the exemplary method of a unified platform for universal healthcare and related services, in accordance with an embodiment of the present invention illustrated in FIG. 20 further includes the operation 2104 of converting raw data inputs into a universally readable format on the system's server and graphic user interface components to facilitate quicker searching and automated comparison of data by the system's machine learning interface whereby the raw data is converted into the universally readable format at an input into the system, while data is in the server, and at an output of the system. The universally readable format may be configured for easy attachment to blockchains to thereby facilitate quicker transmission of the data, whereby the universally readable format includes executable functions to compress and decompress the raw data, wherein the blockchain will not contain raw data or imagery. The universally readable format may exist as compressed metadata that is decoded by the graphic user interface for the specific variable-based panel system and displayed portal.

In some embodiments, the exemplary method of a unified platform for universal healthcare and related services, in accordance with an embodiment of the present invention illustrated in FIG. 20 further includes the operation 2106 of adjusting the recommended treatment and medication of a patient based on the availability of an organ.

In some embodiments, the exemplary method of a unified platform for universal healthcare and related services, in accordance with an embodiment of the present invention illustrated in FIG. 20 further includes the operation 2108 of scheduling appointments with medical practitioners.

In some embodiments, the exemplary method of a unified platform for universal healthcare and related services, in accordance with an embodiment of the present invention illustrated in FIG. 20 further includes the operation 2110 of facilitating a purchase of a medication based on the participant medical record.

In some embodiments, the exemplary method of a unified platform for universal healthcare and related services, in accordance with an embodiment of the present invention illustrated in FIG. 20 further includes the operation 2112 of tracking shipment of the medication and organ based on the participant medical record. The operation 2112 of tracking comprises tracking an organ pick-up location and a delivery of the organ to a destination location.

In some embodiments, the exemplary method of a unified platform for universal healthcare and related services, in accordance with an embodiment of the present invention illustrated in FIG. 20 further includes the operation 2114 of making healthcare related cost information public by displaying the healthcare related cost information in a panel of the portal displayed on the graphic user interface, thereby creating competition within a healthcare industry.

In some embodiments, the exemplary method of a unified platform for universal healthcare and related services, in accordance with an embodiment of the present invention illustrated in FIG. 20 further includes the operation 2116 of defining symptoms and genetics of the participant by implementation of machine learning wherein a machine learning system receives input from an end user and stores the input in a memory of a healthcare computational module that receives and processes the end user input to further produce and communicate suggested care options to the end user at an outcome module, wherein the machine learning system is configured to use medical history to compare to the medical history to participant's medical history, wherein the medical history includes patient's medical data, medical images, and stored medical information including non-patient medical images, thereby resulting in medical diagnostics based on the defined symptoms and genetics of the participant wherein the medical diagnostics is configured to at least partially base diagnostics on the transformed medical records of the blockchain-based software program. The operation 2116 of defining of the symptoms and genetics of the participant by implementation of machine learning further includes identifying lighting and coloration on a pixel-by-pixel basis that would otherwise be undetectable to the naked eye, cataloguing the identifications and comparing at least one identification to at least one medical image to detect anomalies, abnormalities, and differences, and automatically applying a summarized text containing a written diagnosis for a user to read.

In some embodiments, the method as illustrated in FIGS. 20-24 is accomplished by using a non-transitory computer-readable storage medium with an executable program stored thereon, wherein the program instructs one or more processors to perform the following steps of the method.

In some embodiments, the method as illustrated in FIG. 20 is incorporated into a computing system with at least one processor, a graphical user interface, and at least one memory device including instructions embodied thereon, wherein the instructions, which when executed by the one or more processors, cause the processors to perform operations for processing of data in a medical evaluation workflow, wherein the operations comprises the steps according to the method.

It will be further apparent to those skilled in the art that at least a portion of the novel method steps and/or system components of the present invention may be practiced and/or located in location(s) possibly outside the jurisdiction of the United States of America (USA), whereby it will be accordingly readily recognized that at least a subset of the novel method steps and/or system components in the foregoing embodiments must be practiced within the jurisdiction of the USA for the benefit of an entity therein or to achieve an object of the present invention. Thus, some alternate embodiments of the present invention may be configured to comprise a smaller subset of the foregoing means for and/or steps described that the applications designer will selectively decide, depending upon the practical considerations of the particular implementation, to carry out and/or locate within the jurisdiction of the USA.

For example, any of the foregoing described method steps and/or system components which may be performed remotely over a network (e.g., without limitation, a remotely located server) may be performed and/or located outside of the jurisdiction of the USA while the remaining method steps and/or system components (e.g., without limitation, a locally located client) of the forgoing embodiments are typically required to be located/performed in the USA for practical considerations. In client-server architectures, a remotely located server typically generates and transmits required information to a US based client, for use according to the teachings of the present invention. Depending upon the needs of the particular application, it will be readily apparent to those skilled in the art, in light of the teachings of the present invention, which aspects of the present invention can or should be located locally and which can or should be located remotely. Thus, for any claims construction of the following claim limitations that are construed under 35 USC § 112 (6)/(f) it is intended that the corresponding means for and/or steps for carrying out the claimed function are the ones that are locally implemented within the jurisdiction of the USA, while the remaining aspect(s) performed or located remotely outside the USA are not intended to be construed under 35 USC § 112 (6) pre-AIA or 35 USC § 112 (f) post AIA. In some embodiments, the methods and/or system components which may be located and/or performed remotely include, without limitation: any one or more of the operations described in connections of the systems and/or processes of the “A-Medicare” system and/or digital platform.

It is noted that according to USA law, all claims must be set forth as a coherent, cooperating set of limitations that work in functional combination to achieve a useful result as a whole. Accordingly, for any claim having functional limitations interpreted under 35 USC § 112 (6)/(f) where the embodiment in question is implemented as a client-server system with a remote server located outside of the USA, each such recited function is intended to mean the function of combining, in a logical manner, the information of that claim limitation with at least one other limitation of the claim.

For example, in client-server systems where certain information claimed under 35 USC § 112 (6)/(f) is/(are) dependent on one or more remote servers located outside the USA, it is intended that each such recited function under 35 USC § 112 (6)/(f) is to be interpreted as the function of the local system receiving the remotely generated information required by a locally implemented claim limitation, wherein the structures and or steps which enable, and breathe life into the expression of such functions claimed under 35 USC § 112 (6)/(f) are the corresponding steps and/or means located within the jurisdiction of the USA that receive and deliver that information to the client (e.g., without limitation, client-side processing and transmission networks in the USA). When this application is prosecuted or patented under a jurisdiction other than the USA, then “USA” in the foregoing should be replaced with the pertinent country or countries or legal organization(s) having enforceable patent infringement jurisdiction over the present patent application, and “35 USC § 112 (6)/(f)” should be replaced with the closest corresponding statute in the patent laws of such pertinent country or countries or legal organization(s).

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

It is noted that according to USA law 35 USC § 112 (1), all claims must be supported by sufficient disclosure in the present patent specification, and any material known to those skilled in the art need not be explicitly disclosed. However, 35 USC § 112 (6) requires that structures corresponding to functional limitations interpreted under 35 USC § 112 (6) must be explicitly disclosed in the patent specification. Moreover, the USPTO's Examination policy of initially treating and searching prior art under the broadest interpretation of a “mean for” or “steps for” claim limitation implies that the broadest initial search on 35 USC § 112(6) (post AIA 112(f)) functional limitation would have to be conducted to support a legally valid Examination on that USPTO policy for broadest interpretation of “mean for” claims. Accordingly, the USPTO will have discovered a multiplicity of prior art documents including disclosure of specific structures and elements which are suitable to act as corresponding structures to satisfy all functional limitations in the below claims that are interpreted under 35 USC § 112(6) (post AIA 112(f)) when such corresponding structures are not explicitly disclosed in the foregoing patent specification.

Therefore, for any invention element(s)/structure(s) corresponding to functional claim limitation(s), in the below claims interpreted under 35 USC § 112(6) (post AIA 112(f)), which is/are not explicitly disclosed in the foregoing patent specification, yet do exist in the patent and/or non-patent documents found during the course of USPTO searching, Applicant(s) incorporate all such functionally corresponding structures and related enabling material herein by reference for the purpose of providing explicit structures that implement the functional means claimed.

Applicant(s) request(s) that fact finders during any claims construction proceedings and/or examination of patent allowability properly identify and incorporate only the portions of each of these documents discovered during the broadest interpretation search of 35 USC § 112(6) (post AIA 112(f)) limitation, which exist in at least one of the patent and/or non-patent documents found during the course of normal USPTO searching and or supplied to the USPTO during prosecution.

Applicant(s) also incorporate by reference the bibliographic citation information to identify all such documents comprising functionally corresponding structures and related enabling material as listed in any PTO Form-892 or likewise any information disclosure statements (IDS) entered into the present patent application by the USPTO or Applicant(s) or any 3^{rd} parties. Applicant(s) also reserve its right to later amend the present application to explicitly include citations to such documents and/or explicitly include the functionally corresponding structures which were incorporate by reference above.

Thus, for any invention element(s)/structure(s) corresponding to functional claim limitation(s), in the below claims, that are interpreted under 35 USC § 112(6) (post AIA 112(f)), which is/are not explicitly disclosed in the foregoing patent specification, Applicant(s) have explicitly prescribed which documents and material to include the otherwise missing disclosure, and have prescribed exactly which portions of such patent and/or non-patent documents should be incorporated by such reference for the purpose of satisfying the disclosure requirements of 35 USC § 112 (6). Applicant(s) note that all the identified documents above which are incorporated by reference to satisfy 35 USC § 112 (6) necessarily have a filing and/or publication date prior to that of the instant application, and thus are valid prior documents to incorporated by reference in the instant application.

Having fully described at least one embodiment of the present invention, other equivalent or alternative methods of implementing a comprehensive digitally-based healthcare services procurement and delivery solution according to the present invention will be apparent to those skilled in the art. Various aspects of the invention have been described above by way of illustration, and the specific embodiments disclosed are not intended to limit the invention to the particular forms disclosed. The particular implementation of the comprehensive digitally-based healthcare services procurement and delivery solution may vary depending upon the particular context or application. By way of example, and not limitation, the comprehensive digitally-based healthcare services procurement and delivery solution described in the foregoing were principally directed to consumer healthcare related implementations; however, similar techniques may instead be applied to businesses and/or other private and/or non-private entities, which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The Abstract is provided to comply with 37 C.F.R. Section 1.72(b) requiring an abstract that will allow the reader to ascertain the nature and gist of the technical disclosure. That is, the Abstract is provided merely to introduce certain concepts and not to identify any key or essential features of the claimed subject matter. It is submitted with the understanding that it will not be used to limit or interpret the scope or meaning of the claims.

The following claims are hereby incorporated into the detailed description, with each claim standing on its own as a separate embodiment.

Only those claims which employ the words “means for” or “steps for” are to be interpreted under 35 USC 112, sixth paragraph (pre-AIA) or 35 USC 112(f) post-AIA. Otherwise, no limitations from the specification are to be read into any claims, unless those limitations are expressly included in the claims.

What is claimed is:

1. A method comprising the steps of:
providing a blockchain-based software program, stored in a non-transitory computer-readable storage digital medium and being executed by one or more processors of a computer-based system, wherein the blockchain-based software program is configured to perform the following steps of:
storing personal electronic medical records of participants into a healthcare database as a growing list of said personal electronic medical records including personal electronic medical record blocks that are linked together using cryptography, wherein each personal electronic medical record block contains a cryptographic hash of the previous personal electronic medical record block;
wherein once said personal electronic medical is recorded, said personal electronic medical record in any given block cannot be altered retroactively without alteration of all subsequent personal electronic medical record blocks, which requires consensus of a network majority;
storing an organ-donor information into said healthcare database as a growing list of said organ-donor information including organ-donor information blocks that are linked together using cryptography, wherein each organ-donor information block contains a cryptographic hash of the previous organ-donor information block;
wherein once said organ-donor information is recorded, said organ-donor information in any given block cannot be altered retroactively without alteration of all subsequent organ-donor information blocks, which requires consensus of a network majority;
creating an international universal database, wherein said international universal database is configured to connect the organ-donor with a recipient at least nationally or worldwide;
matching a donated organ to a participant based on the stored electronic medical record of the participant and the organ-donor information;
determining a location where the organ will be donated based on said matching step; and
providing a variable-based panel system, including a graphic user interface, where a user can manage at least one of, a doctor's appointment, said electronic medical record, healthcare insurance, hospital visits, medications, and pharmacy, wherein said variable-based panel is configured to be locked with an assigned ID and Social Security Number;
providing a graphic user interface coupled to said non-transitory computer-readable storage digital medium and said processor, whereby said graphic user interface displays a graphical output of said computer-based-system on a screen, including:
displaying an interactive portal defined by graphical buttons represented by interactive locations on said screen;
displaying said graphical buttons represented by said interactive locations, wherein, when engaged, said interactive buttons executes at least one function, including providing a menu of executable sub functions, including displaying a bank of credits, showing advertisements, displaying healthcare information, whereby selection of each option executes commands to query a server, receive real-time information, and populate said information on said screen;
populating advertising content on said screen of said graphic user interface, with a computer ad-based offerings module hardware, at least partially based on successful consumption of the advertising content as indicated by a completion of a survey by the participant and return of same to a private-party advertiser, wherein the participant earns credit in return from consumption of the advertising content;
rewarding, with said computer ad-based offerings module hardware, participants with credit towards the participant's healthcare bills or premiums;
coordinating where an organ of said organ-donor will be donated based on said matching step, the participant medical records, and organ-donor information;
tracking a shipment of the donated organ based on said facilitating step and the stored participant medical record;
wherein said tracking step comprises tracking an organ pick-up location and a delivery of the organ to a destination location;
creating a recycling program for unused medications including the step of rewarding credit to the participant, thereby motivating participants to recycle unused medications to local pharmacies; and
distributing credits, wherein said distributed credits include at least one of said credits obtained for participating in viewing advertising content and said credits obtained from participating in said recycling program, in which at least a portion of the credit is distributed to multiple entities including the user, the medical insurer, the pharmacy, and governmental health agencies, wherein the user's portion of credit is recorded in a virtual bank of credits to be selectably used on medical bills and services.

2. The method as recited in claim 1 wherein said blockchain-based program includes an open, distributed ledger that is configured to record a transaction between said organ-donor and participant including said organ-donor and participant in a verifiable and permanent way.

3. The method as recited in claim 1 wherein said advertising content further includes a digital platform for advertisements where the private party advertiser pays to advertise their products to specific target audiences with the survey at an end of each advertisement.

4. The method as recited in claim 1 wherein said blockchain-based program is further configured to perform the following steps of:
providing a virtual personal health assistant (“VPHA”), wherein said virtual personal health assistant is configured to be operable for following voice and/or text commands to provide information and assistance.

5. The method as recited in claim 4, wherein said virtual personal health assistant is a home-based robot connected to said blockchain-based program through a home portal, and said robot including a memory on which said robot includes instructions thereon for:
receiving voice commands and execute functions based on said commands; and
communicating bi-directionally with an Internet of Things device including at least one of a smart wearable and chip imbedded into a user's skin.

6. The method as recited in claim 1 wherein the blockchain-based program is further configured to perform the steps of;
processing and prioritizing a health insurance claim in at least one of a public or private doctor's office, a public or private specialist, and a public or private hospital or medical facility nationwide.

7. The method as recited in claim 1, further comprising:
sending automated variable-based notifications which are pushed to a user's portal displayed on the screen of the graphic user interface, wherein notifications result from at least real-time changes to a patient's information including conditions reported by a review from machine learning, conditions observed by wearable and Internet of Things technology, availability of organs, and changes in diagnosis.

8. The method as recited in claim 1, further comprising:
converting raw data inputs into a universally readable format on the system's server and graphic user interface components to facilitate quicker searching and automated comparison of data by the system's machine learning interface whereby said raw data is converted into said universally readable format at an input into the system, while data is in said server, and at an output of said system;
said universally readable format configured for easy attachment to blockchains to thereby facilitate quicker transmission of said data, whereby said universally readable format includes executable functions to compress and decompress said raw data, wherein the blockchain will not contain raw data or imagery; and
said universally readable format existing as compressed metadata that is decoded by the graphic user interface for the specific variable-based panel system and displayed portal.

9. The method as recited in claim 1, further comprising:
adjusting the recommended treatment and medication of a patient based on the availability of an organ.

10. A non-transitory computer-readable storage medium with an executable program stored thereon, wherein the program instructs one or more processors to perform the following steps:
storing a blockchain-based software program in a non-transitory digital storage medium and executed by one or more processors of a computer-based system, wherein the blockchain-based software program is configured to perform the following steps:
storing electronic medical records of participants into a healthcare database as a growing list of said personal electronic medical records including personal electronic medical record blocks that are linked together using cryptography, wherein each personal electronic medical record block contains a cryptographic hash of the previous personal electronic medical record block;
wherein once said personal electronic medical is recorded, said personal electronic medical record in any given block cannot be altered retroactively without alteration of all subsequent personal electronic medical record blocks, which requires consensus of a network majority;
storing an organ-donor information into said healthcare database as a growing list of organ-donor information including organ-donor information blocks that are linked together using cryptography, wherein each organ-donor information block contains a cryptographic hash of the previous organ-donor information block;
wherein once said organ-donor information is recorded, said organ-donor information in any given block cannot be altered retroactively without alteration of all subsequent organ-donor information blocks, which requires consensus of a network majority;
scheduling appointments with medical practitioners;
matching a donated organ to a participant based on the stored electronic medical record of the participant and the organ-donor information; and
determining a location where the organ will be donated based on said matching step;
providing a graphic user interface coupled to said non-transitory computer-readable storage digital medium and said processor, whereby said graphic user interface displays a graphical output of said computer-based-system on a screen, including:
displaying an interactive portal defined by graphical buttons represented by interactive locations on said screen;
displaying said graphical buttons represented by said interactive locations, wherein, when engaged, said interactive buttons executes at least one function, including providing a menu of executable sub functions, including displaying a bank of credits, showing advertisements, displaying healthcare information, whereby selection of each option executes commands to query a server, receive real-time information, and populate said information on said screen;
providing an advertising platform, said advertising platform is configured to be operable for generating credits for a participant who view an advertising content on a graphic user interface, wherein the participant earns said generated credits in return from viewing of the advertising content as indicated by a completion of a survey by the participant;
coordinating where an organ of said organ-donor will be donated based on the stored medical records and organ-donor information;
adjusting the recommended treatment and medication of a patient based on the availability of an organ;
facilitating a purchase of a medication based on the participant medical record;
tracking shipment of the medication and organ based on the participant medical record, said tracking step comprises tracking an organ pick-up location and a delivery of the organ to a destination location;
creating a recycling program for unused medication including the step of rewarding credit to the participant thereby motivating participants to recycle unused medications to local pharmacies; and
distributing credits, wherein said distributed credits include at least one of said credits obtained for participating in viewing advertising content and said credits obtained from participating in said recycling program, in which at least a portion of the credit is distributed to multiple entities including the user, the medical insurer, the pharmacy, and governmental health agencies, wherein the user's portion of credit is recorded in a virtual bank of credits to be selectably used on medical bills and services.

11. The program instructing the one or more processors as recited in claim 10, further comprising:
sending automated variable-based notifications which are pushed to a user's portal displayed on the screen of the graphic user interface, wherein notifications result from at least real-time changes to a patient's information including conditions reported by a review from machine learning, conditions observed by wearable and Internet of Things technology, availability of organs, and changes in diagnosis.

12. The program instructing the one or more processors as recited in claim 10, further comprising:
converting raw data inputs into a universally readable format on the system's server and graphic user interface components to facilitate quicker searching and automated comparison of data by the system's machine learning interface whereby said raw data is converted into said universally readable format at an input into the system, while data is in said server, and at an output of said system;
said universally readable format configured for easy attachment to blockchains to thereby facilitate quicker transmission of said data, whereby said universally readable format includes executable functions to compress and decompress said raw data, wherein the blockchain will not contain raw data or imagery; and
said universally readable format existing as compressed metadata that is decoded by the graphic user interface for the specific variable-based panel system and displayed portal.

13. The program instructing the one or more processors as recited in claim 10, further comprising a step of:
making healthcare related cost information public by displaying said healthcare related cost information in a panel of said portal displayed on said graphic user interface, thereby creating competition within a healthcare industry.

14. A computing system, comprising:
at least one or more processors;
a graphic user interface; and
at least one memory device including instructions embodied thereon, wherein the instructions, which when executed by the one or more processors, cause the processors to perform operations for processing of data in a medical evaluation workflow, wherein the operations comprises:
providing a blockchain-based software program, stored in a non-transitory digital storage medium and executed by the one or more processors, wherein the blockchain-based software program is configured to perform the following steps of:
storing medical records of participants into a healthcare database as a growing list of said personal electronic medical records including personal electronic medical record blocks that are linked together using cryptography, wherein each personal electronic medical record block contains a cryptographic hash of the previous personal electronic medical record block;
wherein once said personal electronic medical is recorded, said personal electronic medical record in any given block cannot be altered retroactively without alteration of all subsequent personal electronic medical record blocks, which requires consensus of a network majority;
storing an organ-donor information into said healthcare database as a growing list of organ-donor information including organ-donor information blocks that are linked together using cryptography, wherein each organ-donor information block contains a cryptographic hash of the previous organ-donor information block;
matching a donated organ to a participant based on the stored medical record of the participant and the organ-donor information; and
scheduling an appointment with a medical practitioner based on said matching step;
providing a graphic user interface software program, stored in a non-transitory digital storage medium and executed by the one or more processors, whereby said graphic user interface displays a graphical output of said graphic user interface software program on a screen, including:
displaying an interactive portal defined by graphical buttons represented by interactive locations on said screen;
displaying said graphical buttons represented by said interactive locations, wherein, when engaged, said interactive buttons executes at least one function, including providing a menu of executable sub functions, including displaying a bank of credits, showing advertisements, displaying healthcare information, whereby selection of each option executes commands to query a server, receive real-time information, and populate said information on said screen;
providing an advertising platform, said advertising platform is configured to be operable for generating credits for participants who view an advertising content on said graphic user interface, wherein the participant earns credit for viewing the advertising content as indicated by a completion of a survey by the participant;
coordinating where an organ of said organ-donor will be donated based on said matching step and the stored participant medical record;
facilitating a purchase of a participant medication based at least partially on the participant medical record;
tracking a shipment of the medication based on said facilitating step and the participant medical record;
tracking a shipment of the donated organ based on said coordinating step and the participant medical record;
creating a recycling program for unused medication including the step of rewarding credit to the participant, thereby motivating participants to recycle unused medications to local pharmacies;
rewarding, with said computer ad-based offerings module hardware, participants with credit towards the participant's healthcare bills or premiums; and
distributing credits, wherein said distributed credits include at least one of said credits obtained for participating in viewing advertising content and said credits obtained from participating in said recycling program, in which at least a portion of the credit is distributed to multiple entities including the user, the medical insurer, the pharmacy, and governmental health agencies, wherein the user's portion of credit is recorded in a virtual bank of credits to be selectably used on medical bills and services.

15. The computing system as recited in claim 14, further comprising:
sending automated variable-based notifications which are pushed to a user's portal displayed on the screen of the graphic user interface, wherein notifications result from at least real-time changes to a patient's information including conditions reported by a review from machine learning, conditions observed by wearable and Internet of Things technology, availability of organs, and changes in diagnosis.

16. The computing system of claim 14 wherein the further comprises an operation of:
applying credit earned from the recycling program to medical needs.

17. The computing system of claim 14, further comprising:
converting raw data inputs into a universally readable format on the system's server and graphic user interface components to facilitate quicker searching and automated comparison of data by the system's machine learning interface whereby said raw data is converted into said universally readable format at an input into the system, while data is in said server, and at an output of said system;
said universally readable format configured for easy attachment to blockchains to thereby facilitate quicker transmission of said data, whereby said universally readable format includes executable functions to compress and decompress said raw data, wherein the blockchain will not contain raw data or imagery; and
said universally readable format existing as compressed metadata that is decoded by the graphic user interface for the specific variable-based panel system and displayed portal.

18. The computing system of claim 14 wherein the blockchain-based software program comprises an open, distributed ledger that is configured to record a transaction between two parties including said organ-donor and participant in a verifiable and permanent way.

19. A non-transitory computer-readable storage medium with an executable program stored thereon, wherein the program instructs one or more processors to perform the following steps, consisting of:
storing a blockchain-based software program in a non-transitory digital storage medium and executed by one or more processors of a computer-based system, wherein the blockchain-based software program is configured to perform the following steps:
storing electronic medical records of participants into a healthcare database as a growing list of said personal electronic medical records including personal electronic medical record blocks that are linked together using cryptography, wherein each personal electronic medical record block contains a cryptographic hash of the previous personal electronic medical record block;
wherein once said personal electronic medical is recorded, said personal electronic medical record in any given block cannot be altered retroactively without alteration of all subsequent personal electronic medical record blocks, which requires consensus of a network majority;
storing an organ-donor information into said healthcare database as a growing list of organ-donor information including organ-donor information blocks that are linked together using cryptography, wherein each organ-donor information block contains a cryptographic hash of the previous organ-donor information block;
wherein once said organ-donor information is recorded, said organ-donor information in any given block cannot be altered retroactively without alteration of all subsequent organ-donor information blocks, which requires consensus of a network majority;
scheduling appointments with medical practitioners;
matching a donated organ to a participant based on the stored electronic medical record of the participant and the organ-donor information; and
determining a location where the organ will be donated based on said matching step;
providing a graphic user interface coupled to said non-transitory computer-readable storage digital medium and said processor, whereby said graphic user interface displays a graphical output of said computer-based-system on a screen, including:
displaying an interactive portal defined by graphical buttons represented by interactive locations on said screen;
displaying said graphical buttons represented by said interactive locations, wherein, when engaged, said interactive buttons executes at least one function, including providing a menu of executable sub functions, including displaying a bank of credits, showing advertisements, displaying healthcare information, whereby selection of each option executes commands to query a server, receive real-time information, and populate said information on said screen;
providing an advertising platform, said advertising platform is configured to be operable for generating credits for a participant who view an advertising content, wherein the participant earns said generated credits in return from viewing of the advertising content on a graphic user interface as indicated by a completion of a survey by the participant;
adjusting the recommended treatment and medication of a patient based on the availability of an organ;
coordinating where an organ of said organ-donor will be donated based on the stored medical records and organ-donor information;
facilitating a purchase of a medication based on the participant medical record;
tracking shipment of the medication and organ based on the participant medical record, said tracking step comprises tracking an organ pick-up location and a delivery of the organ to a destination location;
creating a recycling program for unused medication including the step of rewarding credit to the participant thereby motivating participants to recycle unused medications to local pharmacies;
distributing credits, wherein said distributed credits include at least one of said credits obtained for participating in viewing advertising content and said credits obtained from participating in said recycling program, in which at least a portion of the credit is distributed to multiple entities chosen from a group consisting of the user, the medical insurer, the pharmacy, and governmental health agencies, wherein the user's portion of credit is recorded in a virtual bank of credits to be selectably used on medical bills and services;
defining the symptoms and genetics of the participant by implementation of machine learning wherein a machine learning system receives input from an end user and stores said input in a memory of a healthcare computational module that receives and processes the end user input to further produce and communicate suggested care options to said end user at an outcome module, wherein said machine learning system is configured to use medical history to compare to said medical history to participant's medical history, wherein said medical history includes patient's medical data, medical images, and stored medical information including non-patient medical images, thereby resulting in medical diagnostics based on said defined symptoms and genetics of the participant wherein said medical diagnostics is configured to at least partially base diagnostics on the transformed medical records of said blockchain-based software program; and
said defining of the symptoms and genetics of the participant by implementation of machine learning further includes identifying lighting and coloration on a pixel-by-pixel basis that would otherwise be undetectable to the naked eye, cataloguing said identifications and comparing at least one identification to at least one medical image to detect anomalies, abnormalities, and differences, and automatically applying a summarized text containing a written diagnosis for a user to read.
